(19) United States
(12) Patent Application Publication
(10) Pub. No.: US 2007/0293581 A1
(43) Pub. Date: Dec. 20, 2007

(54) Methods for Buccal, Lingual or Sublingual Dosing Regimens of Epinephrine for the Treatment of Allergic Emergencies

(76) Inventor: Malcolm Hill, Solana Beach, CA (US)

Correspondence Address: WILSON SONSINI GOODRICH & ROSATI, 650 PAGE MILL ROAD, PALO ALTO, CA, 94304-1050, US

(21) Appl. No.: 11/758,668

(22) Filed: Jun. 5, 2007

Related U.S. Application Data
(60) Provisional application No. 60/803,968, filed on Jun. 5, 2006.
(60) Provisional application No. 60/803,975, filed on Jun. 5, 2006.

(51) Int. Cl. A61K 31/198

(52) U.S. Cl. 514/649

(57) ABSTRACT

The present invention relates to methods of administering dosage forms which comprise epinephrine, including buccal, lingual, sublingual or transmucosal dosage forms comprising epinephrine for treatment of allergic emergencies, including anaphylaxis. Also provided herein are kits and packaging systems useful in these methods.

### RELATED APPLICATIONS

This application claims the benefit of U.S. Provisional Application Nos. 60/803,968, filed Jun. 5, 2006; and 60/803,975, filed Jun. 5, 2006, which are hereby incorporated by reference in their entireties.

### FIELD OF THE INVENTION

The present invention relates to methods of administering dosage forms which comprise epinephrine, including buccal, lingual, sublingual or transmucosal dosage forms comprising epinephrine for the treatment of allergic emergencies, including anaphylaxis. Also provided herein are kits and packaging systems useful in these methods.

### BACKGROUND OF THE INVENTION

Allergic emergencies, such as anaphylaxis, are a growing concern, given the increasing awareness of members of the public of their frequency and potential severity. Anaphylaxis is a sudden, severe, systemic allergic reaction that can be fatal, in many cases, if left untreated. Anaphylaxis can involve various areas of the body, such as the skin, respiratory tract, gastrointestinal tract, and cardiovascular system. Acute symptoms occur from within about a minute to about two hours after contact with the allergy-causing substance; but in rare instances onset may be delayed by as much as eight hours. Contact with anaphylaxis-inducing agents, and the severity of the resulting anaphylactic reaction, can be extremely unpredictable. Accordingly, allergists recommend that persons who have a personal or family history of anaphylaxis, or a risk of anaphylaxis, be prepared to self-administer emergency treatment at all times. Additionally, adults charged with caring for children who are at risk for anaphylaxis should also be prepared to administer anti-anaphylactic first aid.

The symptoms of anaphylaxis include one or more of the following, generally within about 1 to about 15 minutes of exposure to the antigen: agitation, a feeling of uneasiness, flushing, palpitations, paresthesias, pruritus, throbbing in the ears, coughing, sneezing, urticaria, angioedema, difficulty breathing due to laryngeal edema or bronchospasm, nausea, vomiting, abdominal pain, diarrhea, shock, convulsions, incontinence, unresponsiveness and death. An anaphylactic reaction may include cardiovascular collapse, even in the absence of respiratory symptoms.

According to the Merck Manual, immediate treatment with epinephrine is imperative for the successful treatment of anaphylaxis. Merck Manual, 17^{th} Ed., 1053-1054 (1999). The recommended dose of epinephrine for the treatment of anaphylaxis is about 0.01 mg/Kg: usually about 0.2 to about 0.5 mL of a 1:1000 dilution of epinephrine in a suitable carrier. It is further recommended that, if the symptoms of anaphylaxis persist after the first dose of epinephrine, the patient be treated with epinephrine doses every five minutes after the initial dose until there is resolution of the anaphylactic symptoms or until the onset of epinephrine toxicity. See Leiberman et al., (2005) J. Allergy Clin. Immunol. 115: S483-S523.

Typically, the epinephrine doses are given manually, either subcutaneously or intramuscularly, and in recent years automatic injectors have become an accepted first aid means of delivering epinephrine. It is recommended that persons at risk of anaphylaxis, and persons responsible for children at risk for anaphylaxis, maintain one or more automatic epinephrine injectors in a convenient place at all times.

Given the difficulties associated with manual subcutaneous or intramuscular administration of epinephrine, such as patient apprehension related to injections, a patient's use of antihistamines (e.g., Benadryl®) as the first line of treatment for an allergic emergency in place of the recommended epinephrine therapy due to the patient's apprehension related to injectable epinephrine dosage forms, or the burden of an at risk person having to always maintain an epinephrine injector close at hand, there exists a need in the art for more convenient methods of immediately administering epinephrine to a person undergoing anaphylaxis wherein epinephrine can be quickly administered in a dosage form that obviates the need for injection or epinephrine injectors. There is a further need for dosing regimens directed to the administration of multiple doses of epinephrine to a person in need thereof in a dosage form that obviates the need for injection or epinephrine injectors. There is also a need for kits or packaging systems wherein a series of multiple doses of epinephrine in a dosage form that obviates the need for injection or epinephrine injectors are enclosed in a package having markings or instructions for use in the treatment of anaphylaxis.

The present invention meets the foregoing needs and provides related advantages as well.

### SUMMARY OF THE INVENTION

The present invention meets the foregoing and related needs by providing an improved method of treating allergic emergencies, such as anaphylaxis, with epinephrine in patients where current injectable treatments are not ideal.

Provided herein are methods for treating an allergic emergency comprising the steps of (a) administering a first dose of a buccal, lingual or sublingual dosage form comprising epinephrine; (b) administering a second dose of a buccal, lingual or sublingual dosage form comprising epinephrine; (c) optionally administering a third dose of a buccal, lingual or sublingual dosage form comprising epinephrine; (d) optionally administering a fourth dose of a buccal, lingual or sublingual dosage form comprising epinephrine; and (e) optionally administering a fifth dose of a buccal, lingual or sublingual dosage form comprising epinephrine. In certain embodiments, the methods comprise administering a first and second dose. In other embodiments, the methods comprise administering a first, second and third dose. In still other embodiments, the methods comprise administering a first, second, third and fourth dose. In yet other embodiments, the methods comprise administering a first, second, third, fourth and fifth dose.

In another aspect of the present invention, provided herein are methods for treating an allergic emergency which increase patient compliance with the epinephrine treatment regimen as compared to traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine; (c) optionally administering a third dose of a buccal, lingual or sublingual dosage form comprising epinephrine; (d) optionally administering a fourth dose of a buccal, lingual or sublingual dosage form comprising epinephrine; and (e) optionally administering a fifth dose of a buccal, lingual or sublingual dosage form comprising epinephrine. In certain embodiments, the methods comprise administering a first and second dose. In other embodiments, the methods comprise administering a first, second and third dose. In still other embodiments, the methods comprise administering a first, second, third and fourth dose. In yet other embodiments, the methods comprise administering a first, second, third, fourth and fifth dose.

In still another aspect of the present invention, provided herein are methods for treating an allergic emergency which reduce patient apprehension associated with traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine; (c) optionally administering a third dose of a buccal, lingual or sublingual dosage form comprising epinephrine; (d) optionally administering a fourth dose of a buccal, lingual or sublingual dosage form comprising epinephrine; and (e) optionally administering a fifth dose of a buccal, lingual or sublingual dosage form comprising epinephrine. In certain embodiments, the methods comprise administering a first and second dose. In other embodiments, the methods comprise administering a first, second and third dose. In still other embodiments, the methods comprise administering a first, second, third and fourth dose. In yet other embodiments, the methods comprise administering a first, second, third, fourth and fifth dose.

In yet another aspect of the present invention, provided herein are methods for the treatment of an allergic emergency which eliminate the pain associated with the administration of traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine; (c) optionally administering a third dose of a buccal, lingual or sublingual dosage form comprising epinephrine; (d) optionally administering a fourth dose of a buccal, lingual or sublingual dosage form comprising epinephrine; and (e) optionally administering a fifth dose of a buccal, lingual or sublingual dosage form comprising epinephrine. In certain embodiments, the methods comprise administering a first and second dose. In other embodiments, the methods comprise administering a first, second and third dose. In still other embodiments, the methods comprise administering a first, second, third and fourth dose. In yet other embodiments, the methods comprise administering a first, second, third, fourth and fifth dose.

In certain embodiments, each buccal, lingual or sublingual dosage form comprises an amount of epinephrine that is bioequivalent to about 0.01 mg/Kg of epinephrine administered by intra-muscular administration. In other embodiments, each buccal, lingual or sublingual dosage form comprises an amount of epinephrine that is bioequivalent to about 0.10 mg to about 0.50 mg of epinephrine administered by intra-muscular injection. In one embodiment, each buccal, lingual or sublingual dosage form comprises an amount of epinephrine that is bioequivalent to about 0.10 mg of epinephrine administered by intramuscular injection. In another embodiment, each buccal, lingual or sublingual dosage form comprises an amount of epinephrine that is bioequivalent to about 0.15 mg of epinephrine administered by intramuscular injection. In still another embodiment, each buccal, lingual or sublingual dosage form comprises an amount of epinephrine that is bioequivalent to about 0.30 mg of epinephrine administered by intramuscular injection. In yet another embodiment, each buccal, lingual or sublingual dosage form comprises an amount of epinephrine that is bioequivalent to about 0.45 mg of epinephrine administered by intra-muscular injection. In still yet another embodiment, each buccal, lingual or sublingual dosage form comprises an amount of epinephrine that is bioequivalent to about 0.50 mg of epinephrine administered by intra-muscular injection. In yet other embodiments, each buccal, lingual or sublingual dosage form comprises from about 1 mg to about 100 mg of epinephrine. In still other embodiments, the each buccal, lingual or sublingual dosage form comprises from about 15 mg to about 60 mg of epinephrine.

In certain other embodiments, the dosing regimen comprising the buccal, lingual or sublingual dosage forms comprises an amount of epinephrine that is bioequivalent to about 0.01 mg/Kg of epinephrine administered by intramuscular administration. In other embodiments, the dosing regimen comprising the buccal, lingual or sublingual dosage forms comprises an amount of epinephrine that is bioequivalent to about 0.1 mg to about 0.5 mg of epinephrine administered by intra-muscular injection. In one embodiment, the dosing regimen comprising the buccal, lingual or sublingual dosage forms comprises an amount of epinephrine that is bioequivalent to about 0.10 mg of epinephrine administered by intramuscular injection. In another embodiment, the dosing regimen comprising the buccal, lingual or sublingual dosage forms comprises an amount of epinephrine that is bioequivalent to about 0.15 mg of epinephrine administered by intramuscular injection. In still another embodiment, the dosing regimen comprising the buccal, lingual or sublingual dosage forms comprises an amount of epinephrine that is bioequivalent to about 0.30 mg of epinephrine administered by intra-muscular injection. In yet another embodiment, the dosing regimen comprising the buccal, lingual or sublingual dosage forms comprises an amount of epinephrine that is bioequivalent to about 0.45 mg of epinephrine administered by intramuscular injection. In still yet another embodiment, the dosing regimen comprising the buccal, lingual or sublingual dosage forms comprises an amount of epinephrine that is bioequivalent to about 0.50 mg of epinephrine administered by intramuscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine.

In some embodiments, the buccal, lingual or sublingual dosage forms can be tablets or films. In certain embodiments, the dosage form is a lingual tablet or film. In other certain embodiments, the dosage form is a sublingual tablet or film. In still other embodiments, the dosage form is a buccal tablet or film. In some embodiments, the buccal, lingual or sublingual dosage forms further comprise a pharmaceutically acceptable excipient. In certain embodiments, the pharmaceutically acceptable excipients comprise an absorption enhancer. In other embodiments, the pharmaceutically acceptable excipient is a transmucosal absorption enhancer.

In certain embodiments, the methods comprise administering the dosage forms by the patient. In other embodiments, the dosage forms can be administered to the patient by another person, such as a parent, a guardian, a care giver, or a health care professional. In certain embodiments, such healthcare professionals administer in an emergency setting, such as in the field, including ambulances or at a patient's home, etc.

In some embodiments, the methods comprise a time interval between administrations of each consecutive or sequential dose of between about 3 minutes to about 10 minutes. In other embodiments, the time interval between consecutive or sequential administrations is about 5 minutes.

Also provided herein are methods for treating an allergic emergency comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the second dose; (d) optionally administering a fourth dose of a buccal, lingual or sublingual dosage form comprising epinephrine wherein the amount of epinephrine in the fourth dose is about 50% to about 100% the amount of epinephrine in the third dose; and (e) optionally administering a fifth dose of a buccal, lingual or sublingual dosage form comprising epinephrine wherein the amount of epinephrine in the fifth dose is about 50% to about 100% the amount of epinephrine in the fourth dose. In other embodiments, each subsequent dose can be about 10% to about 100% the amount of epinephrine of the prior dose, e.g. the second dose can be about 10% to about 100% the amount of epinephrine of the first dose. In yet other embodiments, each subsequent dose can be about 30% to about 100% the amount of epinephrine of the prior dose. In still other embodiments, each subsequent dose can be about 40% to about 100% the amount of epinephrine of the prior dose.

In one aspect of the present invention, provided herein are methods for treating an allergic emergency which increase patient compliance with the epinephrine treatment regimen as compared to traditional injectable epinephrine treatment regimens, the methods comprising the steps of comprising the steps of a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the second dose; (d) optionally administering a fourth dose of a buccal, lingual or sublingual dosage form comprising epinephrine wherein the amount of epinephrine in the fourth dose is about 50% to about 100% the amount of epinephrine in the third dose; and (e) optionally administering a fifth dose of a buccal, lingual or sublingual dosage form comprising epinephrine wherein the amount of epinephrine in the fifth dose is about 50% to about 100% the amount of epinephrine in the fourth dose. In other embodiments, each subsequent dose can be about 10% to about 100% the amount of epinephrine of the prior dose, e.g. the second dose can be about 10% to about 100% the amount of epinephrine of the first dose. In yet other embodiments, each subsequent dose can be about 30% to about 100% the amount of epinephrine of the prior dose. In still other embodiments, each subsequent dose can be about 40% to about 100% the amount of epinephrine of the prior dose.

In another aspect of the present invention, provided herein are methods for treating an allergic emergency which reduce patient apprehension associated with traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the second dose; (d) optionally administering a fourth dose of a buccal, lingual or sublingual dosage form comprising epinephrine wherein the amount of epinephrine in the fourth dose is about 50% to about 100% the amount of epinephrine in the third dose; and (e) optionally administering a fifth dose of a buccal, lingual or sublingual dosage form comprising epinephrine wherein the amount of epinephrine in the fifth dose is about 50% to about 100% the amount of epinephrine in the fourth dose. In other embodiments, each subsequent dose can be about 10% to about 100% the amount of epinephrine of the prior dose, e.g. the second dose can be about 10% to about 100% the amount of epinephrine of the first dose. In yet other embodiments, each subsequent dose can be about 30% to about 100% the amount of epinephrine of the prior dose. In still other embodiments, each subsequent dose can be about 40% to about 100% the amount of epinephrine of the prior dose.

In yet another aspect of the present invention, provided herein are methods for the treatment of an allergic emergency which eliminate the pain associated with the administration of traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the second dose; (d) optionally administering a fourth dose of a buccal, lingual or sublingual dosage form comprising epinephrine wherein the amount of epinephrine in the fourth dose is about 50% to about 100% the amount of epinephrine in the third dose; and (e) optionally administering a fifth dose of a buccal, lingual or sublingual dosage form comprising epinephrine wherein the amount of epinephrine in the fifth dose is about 50% to about 100% the amount of epinephrine in the fourth dose. In other embodiments, each subsequent dose can be about 10% to about 100% the amount of epinephrine of the prior dose, e.g. the second dose can be about 10% to about 100% the amount of epinephrine of the first dose. In yet other embodiments, each subsequent dose can be about 30% to about 100% the amount of epinephrine of the prior dose. In still other embodiments, each subsequent dose can be about 40% to about 100% the amount of epinephrine of the prior dose.

In certain embodiments, the first buccal, lingual or sublingual dosage form comprises an amount of epinephrine that is bioequivalent to about 0.01 mg/Kg of epinephrine administered by intra-muscular administration. In other embodiments, the first buccal, lingual or sublingual dosage form comprises an amount of epinephrine that is bioequivalent to about 0.10 mg to about 0.50 mg of epinephrine administered by intra-muscular injection. In one embodiment, the first buccal, lingual or sublingual dosage form comprises an amount of epinephrine that is bioequivalent to about 0.10 mg of epinephrine administered by intramuscular injection. In another embodiment, the first buccal, lingual or sublingual dosage form comprises an amount of epinephrine that is bioequivalent to about 0.15 mg of epinephrine administered by intramuscular injection. In still another embodiment, the first buccal, lingual or sublingual dosage form comprises an amount of epinephrine that is bioequivalent to about 0.30 mg of epinephrine administered by intramuscular injection. In yet another embodiment, the first buccal, lingual or sublingual dosage form comprises an amount of epinephrine that is bioequivalent to about 0.45 mg of epinephrine administered by intra-muscular injection. In still yet another embodiment, the first buccal, lingual or sublingual dosage form comprises an amount of epinephrine that is bioequivalent to about 0.50 mg of epinephrine administered by intramuscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intra-muscular administration. In other embodiments, the subsequent administration of a second or greater buccal, lingual, or sublingual dosage form is bioequivalent to the subsequent administration of a second or greater injectable dosage form comprising about 0.1 mg to about 0.5 mg of epinephrine administered by intra-muscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intramuscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intramuscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intramuscular injection.

In certain other embodiments, the dosing regimen comprising the buccal, lingual or sublingual dosage forms comprises an amount of epinephrine that is bioequivalent to about 0.01 mg/Kg of epinephrine administered by intramuscular administration. In other embodiments, the dosing regimen comprising the buccal, lingual or sublingual dosage forms comprises an amount of epinephrine that is bioequivalent to about 0.1 mg to about 0.5 mg of epinephrine administered by intramuscular injection. In one embodiment, the dosing regimen comprising the buccal, lingual or sublingual dosage forms comprises an amount of epinephrine that is bioequivalent to about 0.10 mg of epinephrine administered by intramuscular injection. In another embodiment, the dosing regimen comprising the buccal, lingual or sublingual dosage forms comprises an amount of epinephrine that is bioequivalent to about 0.15 mg of epinephrine administered by intramuscular injection. In still another embodiment, the dosing regimen comprising the buccal, lingual or sublingual dosage forms comprises an amount of epinephrine that is bioequivalent to about 0.30 mg of epinephrine administered by intramuscular injection. In yet another embodiment, the dosing regimen comprising the buccal, lingual or sublingual dosage forms comprises an amount of epinephrine that is bioequivalent to about 0.45 mg of epinephrine administered by intra-muscular injection. In still yet another embodiment, the dosing regimen comprising the buccal, lingual or sublingual dosage forms comprises an amount of epinephrine that is bioequivalent to about 0.50 mg of epinephrine administered by intra-muscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine. In certain embodiments, two buccal, lingual or sublingual dosage forms comprises an amount of epinephrine that is bioequivalent to about 0.15 mg of epinephrine administered by intra-muscular injection. In certain other embodiments, two buccal, lingual or sublingual dosage forms comprises an amount of epinephrine that is bioequivalent to about 0.30 mg of epinephrine administered by intramuscular injection. In still other embodiments, two buccal, lingual or sublingual dosage forms comprises an amount of epinephrine that is bioequivalent to about 0.50 mg of epinephrine administered by intra-muscular injection. In certain embodiments, three buccal, lingual or sublingual dosage forms comprises an amount of epinephrine that is bioequivalent to about 0.15 mg of epinephrine administered by intramuscular injection. In certain other embodiments, three buccal, lingual or sublingual dosage forms comprises an amount of epinephrine that is bioequivalent to about 0.30 mg of epinephrine administered by intramuscular injection. In still other embodiments, three buccal, lingual or sublingual dosage forms comprises an amount of epinephrine that is bioequivalent to about 0.50 mg of epinephrine administered by intramuscular injection.

In some embodiments, the buccal, lingual or sublingual dosage forms can be tablets or films. In certain embodiments, the dosage form is a lingual tablet or film. In certain other embodiments, the dosage form is a sublingual tablet or film. In still other embodiments, the dosage form is a buccal tablet or film. In some embodiments, the buccal, lingual or sublingual dosage forms further comprise a pharmaceutically acceptable excipient. In certain embodiments, the pharmaceutically acceptable excipients comprise an absorption enhancer. In other embodiments, the pharmaceutically acceptable excipient is a transmucosal absorption enhancer.

In some embodiments, the pharmaceutically acceptable excipient can be an absorption enhancer that is present in only the second dose. In other embodiments, the pharmaceutically acceptable excipient can be an absorption enhancer that is present in only the second and third dose. In still other embodiments, the pharmaceutically acceptable excipient can be an absorption enhancer that is present in only the second, third and fourth dose. In yet other embodiments, the pharmaceutically acceptable excipient can be an absorption enhancer that is present in only the second, third, fourth, and fifth dose.

In certain embodiments, the methods comprise administering the dosage forms by the patient. In other embodiments, the dosage forms can be administered to the patient by another person, such as a parent, a guardian, a care giver, or a health care professional. In certain embodiments, such healthcare professionals administer in an emergency setting, such as in the field, including ambulances or at a patient's home, etc.

In some embodiments, the methods comprise a time interval between administrations of each consecutive or sequential dose of between about 3 minutes to about 10 minutes. In other embodiments, the time interval between consecutive or sequential administrations is about 5 minutes.

Also provided herein are methods for treating an allergic emergency comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the firstdose; (d) optionally administering a fourth dose of a buccal, lingual or sublingual dosage form comprising epinephrine wherein the amount of epinephrine in the fourth dose is about 50% to about 100% the amount of epinephrine in the first dose; and (e) optionally administering a fifth dose of a buccal, lingual or sublingual dosage form comprising epinephrine wherein the amount of epinephrine in the fifth dose is about 50% to about 100% the amount of epinephrine in the first dose. In other embodiments, each subsequent dose can be about 10% to about 100% the amount of epinephrine of the first dose. In yet other embodiments, each subsequent dose can be about 30% to about 100% the amount of epinephrine of the first dose. In still other embodiments, each subsequent dose can be about 40% to about 100% the amount of epinephrine of the first dose.

In one aspect of the present invention, provided herein are methods for treating an allergic emergency which increase patient compliance with the epinephrine treatment regimen as compared to traditional injectable epinephrine treatment regimens, the methods comprising the steps of comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the firstdose; (d) optionally administering a fourth dose of a buccal, lingual or sublingual dosage form comprising epinephrine wherein the amount of epinephrine in the fourth dose is about 50% to about 100% the amount of epinephrine in the first dose; and (e) optionally administering a fifth dose of a buccal, lingual or sublingual dosage form comprising epinephrine wherein the amount of epinephrine in the fifth dose is about 50% to about 100% the amount of epinephrine in the first dose. In other embodiments, each subsequent dose can be about 10% to about 100% the amount of epinephrine of the first dose. In yet other embodiments, each subsequent dose can be about 30% to about 100% the amount of epinephrine of the first dose. In still other embodiments, each subsequent dose can be about 40% to about 100% the amount of epinephrine of the first dose.

In another aspect of the present invention, provided herein are methods for treating an allergic emergency which reduce patient apprehension associated with traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the firstdose; (d) optionally administering a fourth dose of a buccal, lingual or sublingual dosage form comprising epinephrine wherein the amount of epinephrine in the fourth dose is about 50% to about 100% the amount of epinephrine in the first dose; and (e) optionally administering a fifth dose of a buccal, lingual or sublingual dosage form comprising epinephrine wherein the amount of epinephrine in the fifth dose is about 50% to about 100% the amount of epinephrine in the first dose. In other embodiments, each subsequent dose can be about 10% to about 100% the amount of epinephrine of the first dose. In yet other embodiments, each subsequent dose can be about 30% to about 100% the amount of epinephrine of the first dose. In still other embodiments, each subsequent dose can be about 40% to about 100% the amount of epinephrine of the first dose.

In yet another aspect of the present invention, provided herein are methods for the treatment of an allergic emergency which eliminate the pain associated with the administration of traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the firstdose; (d) optionally administering a fourth dose of a buccal, lingual or sublingual dosage form comprising epinephrine wherein the amount of epinephrine in the fourth dose is about 50% to about 100% the amount of epinephrine in the first dose; and (e) optionally administering a fifth dose of a buccal, lingual or sublingual dosage form comprising epinephrine wherein the amount of epinephrine in the fifth dose is about 50% to about 100% the amount of epinephrine in the first dose. In other embodiments, each subsequent dose can be about 10% to about 100% the amount of epinephrine of the first dose. In yet other embodiments, each subsequent dose can be about 30% to about 100% the amount of epinephrine of the first dose. In still other embodiments, each subsequent dose can be about 40% to about 100% the amount of epinephrine of the first dose.

In certain embodiments, the first buccal, lingual or sublingual dosage form comprises an amount of epinephrine that is bioequivalent to about 0.01 mg/Kg of epinephrine administered by intra-muscular administration. In other embodiments, the first buccal, lingual or sublingual dosage form comprises an amount of epinephrine that is bioequivalent to about 0.10 mg to about 0.50 mg of epinephrine administered by intramuscular injection. In one embodiment, the first buccal, lingual or sublingual dosage form comprises an amount of epinephrine that is bioequivalent to about 0.10 mg of epinephrine administered by intra-muscular injection. In another embodiment, the first buccal, lingual or sublingual dosage form comprises an amount of epinephrine that is bioequivalent to about 0.15 mg of epinephrine administered by intra-muscular injection. In still another embodiment, the first buccal, lingual or sublingual dosage form comprises an amount of epinephrine that is bioequivalent to about 0.30 mg of epinephrine administered by intra-muscular injection. In yet another embodiment, the first buccal, lingual or sublingual dosage form comprises an amount of epinephrine that is bioequivalent to about 0.45 mg of epinephrine administered by intramuscular injection. In still yet another embodiment, the first buccal, lingual or sublingual dosage form comprises an amount of epinephrine that is bioequivalent to about 0.50 mg of epinephrine administered by intramuscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intramuscular administration. In other embodiments, the subsequent administration of a second or greater buccal, lingual, or sublingual dosage form is bioequivalent to the subsequent administration of a second or greater injectable dosage form comprising about 0.1 mg to about 0.5 mg of epinephrine administered by intramuscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intramuscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intra-muscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intra-muscular injection.

In certain other embodiments, the dosing regimen comprising the buccal, lingual or sublingual dosage forms comprises an amount of epinephrine that is bioequivalent to about 0.01 mg/Kg of epinephrine administered by intra-muscular administration. In other embodiments, the dosing regimen comprising the buccal, lingual or sublingual dosage forms comprises an amount of epinephrine that is bioequivalent to about 0.1 mg to about 0.5 mg of epinephrine administered by intra-muscular injection. In one embodiment, the dosing regimen comprising the buccal, lingual or sublingual dosage forms comprises an amount of epinephrine that is bioequivalent to about 0.10 mg of epinephrine administered by intramuscular injection. In another embodiment, the dosing regimen comprising the buccal, lingual or sublingual dosage forms comprises an amount of epinephrine that is bioequivalent to about 0.15 mg of epinephrine administered by intra-muscular injection. In still another embodiment, the dosing regimen comprising the buccal, lingual or sublingual dosage forms comprises an amount of epinephrine that is bioequivalent to about 0.30 mg of epinephrine administered by intra-muscular injection. In yet another embodiment, the dosing regimen comprising the buccal, lingual or sublingual dosage forms comprises an amount of epinephrine that is bioequivalent to about 0.45 mg of epinephrine administered by intra-muscular injection. In still yet another embodiment, the dosing regimen comprising the buccal, lingual or sublingual dosage forms comprises an amount of epinephrine that is bioequivalent to about 0.50 mg of epinephrine administered by intramuscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine. In certain embodiments, two buccal, lingual or sublingual dosage forms comprises an amount of epinephrine that is bioequivalent to about 0.15 mg of epinephrine administered by intramuscular injection. In certain other embodiments, two buccal, lingual or sublingual dosage forms comprises an amount of epinephrine that is bioequivalent to about 0.30 mg of epinephrine administered by intramuscular injection. In still other embodiments, two buccal, lingual or sublingual dosage forms comprises an amount of epinephrine that is bioequivalent to about 0.50 mg of epinephrine administered by intra-muscular injection. In certain embodiments, three buccal, lingual or sublingual dosage forms comprises an amount of epinephrine that is bioequivalent to about 0.15 mg of epinephrine administered by intra-muscular injection. In certain other embodiments, three buccal, lingual or sublingual dosage forms comprises an amount of epinephrine that is bioequivalent to about 0.30 mg of epinephrine administered by intramuscular injection. In still other embodiments, three buccal, lingual or sublingual dosage forms comprises an amount of epinephrine that is bioequivalent to about 0.50 mg of epinephrine administered by intramuscular injection.

In some embodiments, the buccal, lingual or sublingual dosage forms can be tablets or films. In certain embodiments, the dosage form is a lingual tablet or film. In certain other embodiments, the dosage form is a sublingual tablet or film. In still other embodiments, the dosage form is a buccal tablet or film. In some embodiments, the buccal, lingual or sublingual dosage forms further comprise a pharmaceutically acceptable excipient. In certain embodiments, the pharmaceutically acceptable excipients comprise an absorption enhancer. In other embodiments, the pharmaceutically acceptable excipient is a transmucosal absorption enhancer.

In some embodiments, the pharmaceutically acceptable excipient can be an absorption enhancer that is present in only the second dose. In other embodiments, the pharmaceutically acceptable excipient can be an absorption enhancer that is present in only the second and third dose. In still other embodiments, the pharmaceutically acceptable excipient can be an absorption enhancer that is present in only the second, third and fourth dose. In yet other embodiments, the pharmaceutically acceptable excipient can be an absorption enhancer that is present in only the second, third, fourth, and fifth dose.

In certain embodiments, the methods comprise administering the dosage forms by the patient. In other embodiments, the dosage forms can be administered to the patient by another person, such as a parent, a guardian, a care giver, or a health care professional. In certain embodiments, such healthcare professionals administer in an emergency setting, such as in the field, including ambulances or at a patient's home, etc.

In some embodiments, the methods comprise a time interval between administrations of each consecutive or sequential dose of between about 3 minutes to about 10 minutes. In other embodiments, the time interval between consecutive or sequential administrations is about 5 minutes.

Also provided herein are methods for treating an allergic emergency comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the first dose; (d) optionally administering a fourth dose of a buccal, lingual or sublingual dosage form comprising epinephrine wherein the amount of epinephrine in the fourth dose is about 75% to about 125% the amount of epinephrine in the first dose; and (e) optionally administering a fifth dose of a buccal, lingual or sublingual dosage form comprising epinephrine wherein the amount of epinephrine in the fifth dose is about 75% to about 125% the amount of epinephrine in the first dose.

In one aspect of the present invention, provided herein are methods for treating an allergic emergency which increase patient compliance with the epinephrine treatment regimen as compared to traditional injectable epinephrine treatment regimens, the methods comprising the steps of comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the first dose; (d) optionally administering a fourth dose of a buccal, lingual or sublingual dosage form comprising epinephrine wherein the amount of epinephrine in the fourth dose is about 75% to about 125% the amount of epinephrine in the first dose; and (e) optionally administering a fifth dose of a buccal, lingual or sublingual dosage form comprising epinephrine wherein the amount of epinephrine in the fifth dose is about 75% to about 125% the amount of epinephrine in the first dose.

In another aspect of the present invention, provided herein are methods for treating an allergic emergency which reduce patient apprehension associated with traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the first dose; (d) optionally administering a fourth dose of a buccal, lingual or sublingual dosage form comprising epinephrine wherein the amount of epinephrine in the fourth dose is about 75% to about 125% the amount of epinephrine in the first dose; and (e) optionally administering a fifth dose of a buccal, lingual or sublingual dosage form comprising epinephrine wherein the amount of epinephrine in the fifth dose is about 75% to about 125% the amount of epinephrine in the first dose.

In yet another aspect of the present invention, provided herein are methods for the treatment of an allergic emergency which eliminate the pain associated with the administration of traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the first dose; (d) optionally administering a fourth dose of a buccal, lingual or sublingual dosage form comprising epinephrine wherein the amount of epinephrine in the fourth dose is about 75% to about 125% the amount of epinephrine in the first dose; and (e) optionally administering a fifth dose of a buccal, lingual or sublingual dosage form comprising epinephrine wherein the amount of epinephrine in the fifth dose is about 75% to about 125% the amount of epinephrine in the first dose.

In certain embodiments, the first buccal, lingual or sublingual dosage form comprises an amount of epinephrine that is bioequivalent to about 0.01 mg/Kg of epinephrine administered by intra-muscular administration. In other embodiments, the first buccal, lingual or sublingual dosage form comprises an amount of epinephrine that is bioequivalent to about 0.10 mg to about 0.50 mg of epinephrine administered by intramuscular injection. In one embodiment, the first buccal, lingual or sublingual dosage form comprises an amount of epinephrine that is bioequivalent to about 0.10 mg of epinephrine administered by intramuscular injection. In another embodiment, the first buccal, lingual or sublingual dosage form comprises an amount of epinephrine that is bioequivalent to about 0.15 mg of epinephrine administered by intramuscular injection. In still another embodiment, the first buccal, lingual or sublingual dosage form comprises an amount of epinephrine that is bioequivalent to about 0.30 mg of epinephrine administered by intramuscular injection. In yet another embodiment, the first buccal, lingual or sublingual dosage form comprises an amount of epinephrine that is bioequivalent to about 0.45 mg of epinephrine administered by intra-muscular injection. In still yet another embodiment, the first buccal, lingual or sublingual dosage form comprises an amount of epinephrine that is bioequivalent to about 0.50 mg of epinephrine administered by intramuscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intramuscular administration. In other embodiments, the subsequent administration of a second or greater buccal, lingual, or sublingual dosage form is bioequivalent to the subsequent administration of a second or greater injectable dosage form comprising about 0.1 mg to about 0.5 mg of epinephrine administered by intramuscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intramuscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intramuscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intra-muscular injection.

In certain other embodiments, the dosing regimen comprising the buccal, lingual or sublingual dosage forms comprises an amount of epinephrine that is bioequivalent to about 0.01 mg/Kg of epinephrine administered by intramuscular administration. In other embodiments, the dosing regimen comprising the buccal, lingual or sublingual dosage forms comprises an amount of epinephrine that is bioequivalent to about 0.1 mg to about 0.5 mg of epinephrine administered by intramuscular injection. In one embodiment, the dosing regimen comprising the buccal, lingual or sublingual dosage forms comprises an amount of epinephrine that is bioequivalent to about 0.10 mg of epinephrine administered by intramuscular injection. In another embodiment, the dosing regimen comprising the buccal, lingual or sublingual dosage forms comprises an amount of epinephrine that is bioequivalent to about 0.15 mg of epinephrine administered by intramuscular injection. In still another embodiment, the dosing regimen comprising the buccal, lingual or sublingual dosage forms comprises an amount of epinephrine that is bioequivalent to about 0.30 mg of epinephrine administered by intramuscular injection. In yet another embodiment, the dosing regimen comprising the buccal, lingual or sublingual dosage forms comprises an amount of epinephrine that is bioequivalent to about 0.45 mg of epinephrine administered by intramuscular injection. In still yet another embodiment, the dosing regimen comprising the buccal, lingual or sublingual dosage forms comprises an amount of epinephrine that is bioequivalent to about 0.50 mg of epinephrine administered by intramuscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine. In certain embodiments, two buccal, lingual or sublingual dosage forms comprises an amount of epinephrine that is bioequivalent to about 0.15 mg of epinephrine administered by intramuscular injection. In certain other embodiments, two buccal, lingual or sublingual dosage forms comprises an amount of epinephrine that is bioequivalent to about 0.30 mg of epinephrine administered by intra-muscular injection. In still other embodiments, two buccal, lingual or sublingual dosage forms comprises an amount of epinephrine that is bioequivalent to about 0.50 mg of epinephrine administered by intramuscular injection. In certain embodiments, three buccal, lingual or sublingual dosage forms comprises an amount of epinephrine that is bioequivalent to about 0.15 mg of epinephrine administered by intra-muscular injection. In certain other embodiments, three buccal, lingual or sublingual dosage forms comprises an amount of epinephrine that is bioequivalent to about 0.30 mg of epinephrine administered by intra-muscular injection. In still other embodiments, three buccal, lingual or sublingual dosage forms comprises an amount of epinephrine that is bioequivalent to about 0.50 mg of epinephrine administered by intra-muscular injection.

In some embodiments, the buccal, lingual or sublingual dosage forms can be tablets or films. In certain embodiments, the dosage form is a lingual tablet or film. In certain other embodiments, the dosage form is a sublingual tablet or film. In still other embodiments, the dosage form is a buccal tablet or film. In some embodiments, the buccal, lingual or sublingual dosage forms further comprise a pharmaceutically acceptable excipient. In certain embodiments, the pharmaceutically acceptable excipients comprise an absorption enhancer. In other embodiments, the pharmaceutically acceptable excipient is a transmucosal absorption enhancer.

In some embodiments, the pharmaceutically acceptable excipient can be an absorption enhancer that is present in only the second dose. In other embodiments, the pharmaceutically acceptable excipient can be an absorption enhancer that is present in only the second and third dose. In still other embodiments, the pharmaceutically acceptable excipient can be an absorption enhancer that is present in only the second, third and fourth dose. In yet other embodiments, the pharmaceutically acceptable excipient can be an absorption enhancer that is present in only the second, third, fourth, and fifth dose.

In certain embodiments, the methods comprise administering the dosage forms by the patient. In other embodiments, the dosage forms can be administered to the patient by another person, such as a parent, a guardian, a care giver, or a health care professional. In certain embodiments, such healthcare professionals administer in an emergency setting, such as in the field, including ambulances or at a patient's home, etc.

In some embodiments, the methods comprise a time interval between administrations of each consecutive or sequential dose of between about 3 minutes to about 10 minutes. In other embodiments, the time interval between consecutive or sequential administrations is about 5 minutes.

Also provided herein are methods for treating an allergic emergency comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the second dose; (d) optionally administering a fourth dose of a buccal, lingual or sublingual dosage form comprising epinephrine wherein the amount of epinephrine in the fourth dose is about 75% to about 125% the amount of epinephrine in the third dose; and (e) optionally administering a fifth dose of a buccal, lingual or sublingual dosage form comprising epinephrine wherein the amount of epinephrine in the fifth dose is about 75% to about 125% the amount of epinephrine in the fourth dose.

In one aspect of the present invention, provided herein are methods for treating an allergic emergency which increase patient compliance with the epinephrine treatment regimen as compared to traditional injectable epinephrine treatment regimens, the methods comprising the steps of comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the second dose; (d) optionally administering a fourth dose of a buccal, lingual or sublingual dosage form comprising epinephrine wherein the amount of epinephrine in the fourth dose is about 75% to about 125% the amount of epinephrine in the third dose; and (e) optionally administering a fifth dose of a buccal, lingual or sublingual dosage form comprising epinephrine wherein the amount of epinephrine in the fifth dose is about 75% to about 125% the amount of epinephrine in the fourth dose.

In another aspect of the present invention, provided herein are methods for treating an allergic emergency which reduce patient apprehension associated with traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the second dose; (d) optionally administering a fourth dose of a buccal, lingual or sublingual dosage form comprising epinephrine wherein the amount of epinephrine in the fourth dose is about 75% to about 125% the amount of epinephrine in the third dose; and (e) optionally administering a fifth dose of a buccal, lingual or sublingual dosage form comprising epinephrine wherein the amount of epinephrine in the fifth dose is about 75% to about 125% the amount of epinephrine in the fourth dose.

In yet another aspect of the present invention, provided herein are methods for the treatment of an allergic emergency which eliminate the pain associated with the administration of traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the second dose; (d) optionally administering a fourth dose of a buccal, lingual or sublingual dosage form comprising epinephrine wherein the amount of epinephrine in the fourth dose is about 75% to about 125% the amount of epinephrine in the third dose; and (e) optionally administering a fifth dose of a buccal, lingual or sublingual dosage form comprising epinephrine wherein the amount of epinephrine in the fifth dose is about 75% to about 125% the amount of epinephrine in the fourth dose.

In certain embodiments, the first buccal, lingual or sublingual dosage form comprises an amount of epinephrine that is bioequivalent to about 0.01 mg/Kg of epinephrine administered by intramuscular administration. In other embodiments, the first buccal, lingual or sublingual dosage form comprises an amount of epinephrine that is bioequivalent to about 0.10 mg to about 0.50 mg of epinephrine administered by intramuscular injection. In one embodiment, the first buccal, lingual or sublingual dosage form comprises an amount of epinephrine that is bioequivalent to about 0.10 mg of epinephrine administered by intramuscular injection. In another embodiment, the first buccal, lingual or sublingual dosage form comprises an amount of epinephrine that is bioequivalent to about 0.15 mg of epinephrine administered by intra-muscular injection. In still another embodiment, the first buccal, lingual or sublingual dosage form comprises an amount of epinephrine that is bioequivalent to about 0.30 mg of epinephrine administered by intramuscular injection. In yet another embodiment, the first buccal, lingual or sublingual dosage form comprises an amount of epinephrine that is bioequivalent to about 0.45 mg of epinephrine administered by intramuscular injection. In still yet another embodiment, the first buccal, lingual or sublingual dosage form comprises an amount of epinephrine that is bioequivalent to about 0.50 mg of epinephrine administered by intramuscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intramuscular administration. In other embodiments, the subsequent administration of a second or greater buccal, lingual, or sublingual dosage form is bioequivalent to the subsequent administration of a second or greater injectable dosage form comprising about 0.1 mg to about 0.5 mg of epinephrine administered by intra-muscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intramuscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intra-muscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intramuscular injection.

In certain other embodiments, the dosing regimen comprising the buccal, lingual or sublingual dosage forms comprises an amount of epinephrine that is bioequivalent to about 0.01 mg/Kg of epinephrine administered by intramuscular administration. In other embodiments, the dosing regimen comprising the buccal, lingual or sublingual dosage forms comprises an amount of epinephrine that is bioequivalent to about 0.10 mg to about 0.50 mg of epinephrine administered by intramuscular injection. In one embodiment, the dosing regimen comprising the buccal, lingual or sublingual dosage forms comprises an amount of epinephrine that is bioequivalent to about 0.10 mg of epinephrine administered by intramuscular injection. In another embodiment, the dosing regimen comprising the buccal, lingual or sublingual dosage forms comprises an amount of epinephrine that is bioequivalent to about 0.15 mg of epinephrine administered by intra-muscular injection. In still another embodiment, the dosing regimen comprising the buccal, lingual or sublingual dosage forms comprises an amount of epinephrine that is bioequivalent to about 0.30 mg of epinephrine administered by intramuscular injection. In yet another embodiment, the dosing regimen comprising the buccal, lingual or sublingual dosage forms comprises an amount of epinephrine that is bioequivalent to about 0.45 mg of epinephrine administered by intramuscular injection. In still yet another embodiment, the dosing regimen comprising the buccal, lingual or sublingual dosage forms comprises an amount of epinephrine that is bioequivalent to about 0.50 mg of epinephrine administered by intra-muscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine. In certain embodiments, two buccal, lingual or sublingual dosage forms comprises an amount of epinephrine that is bioequivalent to about 0.15 mg of epinephrine administered by intra-muscular injection. In certain other embodiments, two buccal, lingual or sublingual dosage forms comprises an amount of epinephrine that is bioequivalent to about 0.30 mg of epinephrine administered by intra-muscular injection. In still other embodiments, two buccal, lingual or sublingual dosage forms comprises an amount of epinephrine that is bioequivalent to about 0.50 mg of epinephrine administered by intra-muscular injection. In certain embodiments, three buccal, lingual or sublingual dosage forms comprises an amount of epinephrine that is bioequivalent to about 0.15 mg of epinephrine administered by intra-muscular injection. In certain other embodiments, three buccal, lingual or sublingual dosage forms comprises an amount of epinephrine that is bioequivalent to about 0.30 mg of epinephrine administered by intramuscular injection. In still other embodiments, three buccal, lingual or sublingual dosage forms comprises an amount of epinephrine that is bioequivalent to about 0.50 mg of epinephrine administered by intra-muscular injection.

In some embodiments, the buccal, lingual or sublingual dosage forms can be tablets or films. In certain embodiments, the dosage form is a lingual tablet or film. In certain other embodiments, the dosage form is a sublingual tablet or film. In still other embodiments, the dosage form is a buccal tablet or film. In some embodiments, the buccal, lingual or sublingual dosage forms further comprise a pharmaceutically acceptable excipient. In certain embodiments, the pharmaceutically acceptable excipients comprise an absorption enhancer. In other embodiments, the pharmaceutically acceptable excipient is a transmucosal absorption enhancer.

In some embodiments, the pharmaceutically acceptable excipient can be an absorption enhancer that is present in only the second dose. In other embodiments, the pharmaceutically acceptable excipient can be an absorption enhancer that is present in only the second and third dose. In still other embodiments, the pharmaceutically acceptable excipient can be an absorption enhancer that is present in only the second, third and fourth dose. In yet other embodiments, the pharmaceutically acceptable excipient can be an absorption enhancer that is present in only the second, third, fourth, and fifth dose.

In certain embodiments, the methods comprise administering the dosage forms by the patient. In other embodiments, the dosage forms can be administered to the patient by another person, such as a parent, a guardian, a care giver, or a health care professional. In certain embodiments, such healthcare professionals administer in an emergency setting, such as in the field, including ambulances or at a patient's home, etc.

In some embodiments, the methods comprise a time interval between administrations of each consecutive or sequential dose of between about 3 minutes to about 10 minutes. In other embodiments, the time interval between consecutive or sequential administrations is about 5 minutes.

The invention can further provide a kit or packaging system for treatment of allergic emergencies, such as anaphylaxis, comprising two or more doses of a buccal, lingual or sublingual dosage form comprising epinephrine. In certain embodiments, the kit or packaging system can further comprise instructions for the administration of the two or more doses of a buccal, lingual or sublingual dosage form comprising epinephrine. In some embodiments, the kit or packaging system can comprise two or more doses of a buccal, lingual or sublingual dosage form comprising epinephrine contained within a protective packaging which prevents damage due to moisture, light or exposure to oxygen. In one embodiment, the protective packaging comprises a polymer-lined foil. In still other embodiments, the two or more doses of a buccal, lingual or sublingual dosage form comprising epinephrine are identified by numerical markings or by sequential location within the protective packaging. In another embodiment, the two or more doses of a buccal, lingual or sublingual dosage form comprising epinephrine are further identified in the kit or packaging system by the shape or color of the dosage form. In yet other embodiments, the kit or packaging system can further comprise a carrying case.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1: FIG. 1 provides a three-dimensional view of a blister package comprising five sublingual dosage forms comprising epinephrine as set forth herein. In this embodiment, each sublingual dosage form is identified by numerical marking embossed onto the blister package.

FIG. 2(a) and (b): FIG. 2(a) provides elevated view and 2 (b) provides a horizontal view of a blister package comprising five sublingual dosage forms comprising epinephrine as set forth herein. In this embodiment, each sublingual dosage form is identified by numerical marking embossed onto the blister package.

FIG. 3: FIG. 3 provides a three dimensional view of a kit comprising five buccal dosage forms comprising epinephrine housed within a portable carrying case. In this embodiment, each buccal dosage form is identified by numerical marking embossed onto the blister package. This view further illustrates the top of the carrying case being in the open position so that the buccal dosages forms are visible.

FIG. 4: provides a three-dimensional view of a blister package comprising five sublingual dosage forms comprising epinephrine as set forth herein. In this embodiment, each sublingual dosage form is identified by numerical marking etched onto the dosage form and numerical markings embossed onto the blister package.

FIG. 5: FIG. 5 provides an illustration of one embodiment of a kit or packaging system as described herein which comprises: (a) five (5) buccal dosage forms comprising epinephrine housed in a blister package which is peelably secured to the inside of the packaging system, wherein each buccal dosage form is identified by numerical marking embossed onto the blister package; and (b) written instructions for a patient containing information related to the administration of the buccal and injectable dosage forms housed within the kit.

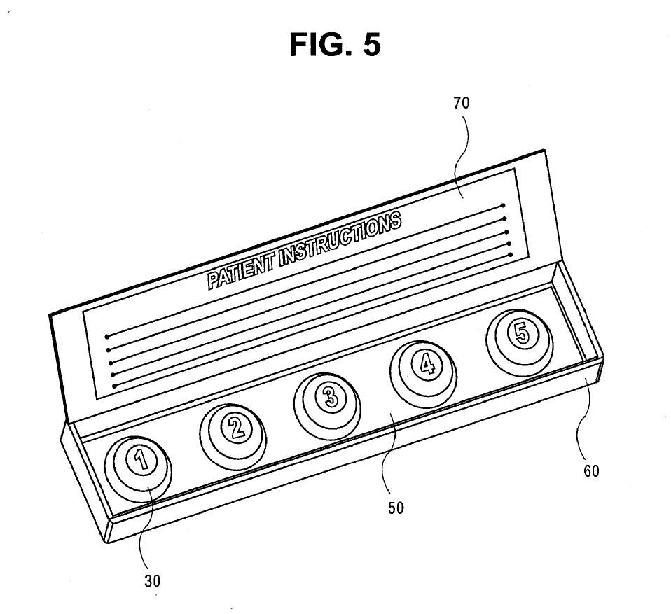

### DETAILED DESCRIPTION OF THE INVENTION

The present invention provides methods for treating allergic emergencies, such as anaphylaxis. The invention further provides buccal, lingual or sublingual dosing regimens of epinephrine for treating allergic emergencies, such as anaphylaxis. Furthermore the invention provides kits or packaging systems comprising buccal, lingual or sublingual dosage forms of epinephrine for treating allergic emergencies, such as anaphylaxis.

As used herein, the term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, are intended to be included within the cited values.

As used herein, the terms “comprising,” “including,” “such as,” and “for example” are used in their open, non-limiting sense.

As used herein, “bioequivalent” or “bioequivalency” refers to one type of dosage form of a certain dose, e.g. buccal, lingual or sublingual comprising about 1 mg to about 100 mg of epinephrine, having the same rate and extent of drug delivery as another type of dosage form at a certain dose, e.g., intramuscular injection of 0.3 mg of epinephrine. Bioequivalence can be shown by any method known in the art of pharmacodynamics or pharmacokinetics, and includes, but is not limited to, studies demonstrating that there is no significant difference between one type of dosage form and another type of dosage form for the mean maximal drug concentration (Cₘₐₓ), the drug concentration time curve (AUC), or the time to maximum concentration in the blood (Tₘₐₓ). In certain aspects of the present invention, bioequivalency can be established by studies which demonstrate that there is no significant difference between one type of dosage form and another type of dosage form with regard to the mean maximal drug concentration (Cₘₐₓ) and the drug concentration time curve (AUC). In certain other aspects of the present invention, bioequivalency can be established by studies which demonstrate that there is no significant difference between one type of dosage form and another type of dosage form with regard any one parameter of pharmacodynamics or pharmacokinetics, including, but not limited to, the mean maximal drug concentration (Cₘₐₓ), the drug concentration time curve (AUC), or the time to maximum concentration in the blood (Tₘₐₓ).

“Therapeutic effect,” as used herein, refers to the amelioration, prevention, inhibition, relief, or termination of any of the symptoms of an allergic emergency described herein.

As used herein, “transmucosal drug delivery,” refers to the delivery of a pharmaceutically active agent through the epithelium for either local or systemic treatment. In some embodiments, transmucosal drug delivery comprises buccal delivery of epinephrine. In certain embodiments, the transmucosal drug delivery comprises a lingual delivery of epinephrine. In certain other embodiments, transmucosal drug delivery comprises sublingual delivery of epinephrine. In still other embodiments, transmucosal drug delivery comprises rectal delivery of epinephrine.

As used herein, “buccal dosage forms,” refer to dosage forms which provide transmucosal delivery of an active agent, e.g., epinephrine, primarily through the epithelial cells of the oral cavity, e.g., the cheek. Buccal dosage forms are known in the art and can include, but are not limited to, patches, lozenges, tablets, oral dissolving/disintegrating tablets (ODTs), muco-adhesive tablets (including muco-adhesive films), fast-melt dissolving tablets (including fast-melt dissolving films), and the like.

As used herein, “lingual dosage forms,” which provide transmucosal delivery of an active agent, e.g., epinephrine, primarily through the oral epithelium. Lingual dosage forms are known in the art and can include, but are not limited to, lozenges, tablets, oral dissolving/disintegrating tablets (ODTs), fast-melt dissolving tablets (including fast-melt dissolving films), orally disintegrating dosage forms, troches, and the like.

As used herein, “sublingual dosage forms,” refer to dosage forms which provide transmucosal delivery of an active agent, e.g., epinephrine, primarily through the oral epithelium beneath the tongue. Sublingual dosage forms are known in the art and can include, but are not limited to, lozenges, tablets, oral dissolving/disintegrating tablets (ODTs), muco-adhesive tablets (including muco-adhesive films), fast-melt dissolving tablets (including fast-melt dissolving films), orally disintegrating dosage forms, troches, and the like.

As used herein, buccal, lingual and sublingual dosage forms refer to oral dosage forms wherein the primary route of administration for the active agent is via the epithelial lining of the oral cavity, e.g., the epithelial cells of the cheek and beneath the tongue. However, it is to be understood that small amounts of active agents delivered by such dosage forms may be swallowed by the patient and absorbed outside the oral cavity.

As used herein, “rectal dosage forms,” refer to dosage forms which provide transmucosal delivery of an active agent, e.g., epinephrine, through the epithelial cells of the rectal cavity. Rectal dosage forms are known in the art and can include, but are not limited to, suppositories, rectal capsules, and gels, creams, and ointments.

As described above, anaphylaxis means an acute and severe allergic reaction to an allergen or an antigen. Treatment of anaphylaxis means at least partially ameliorating or alleviating the symptoms of anaphylaxis. Such treatment may be, and in most cases is, temporary. For example, in certain embodiments of the present invention, the methods, buccal, lingual or sublingual dosing regimens, or kits or packaging systems comprising buccal, lingual or sublingual dosage forms of epinephrine of the invention will provide emergency relief from the symptoms of anaphylaxis for a time sufficient for the patient to seek professional medical assistance. Thus, the methods, buccal, lingual or sublingual dosing regimens, and kits or packaging systems of the invention are well suited for inclusion in first aid kits in professional child care settings and homes, especially where one or more persons at risk for anaphylaxis are known to dwell. They are also well suited for inclusion in so-called crash carts in ambulances or other emergency vehicles, as well as medical emergency rooms. They may also be conveniently carried by those who are at risk for anaphylaxis or those who are charged with caring for those who are at risk for anaphylaxis. The methods of the invention are suitable for treating persons who are at risk for allergic emergencies, such as anaphylaxis, in any of the aforementioned settings.

Thus, treatment of an allergic emergency includes treatment of anaphylaxis, for which the invention is especially well-suited. In addition, treatment of allergic emergency includes treatment of other allergic conditions that may be treated with epinephrine. For example, the symptoms of anaphylactoid reactions to drugs closely mimic those of anaphylaxis and are treated in a similar manner. In cases where it is not clear whether the reaction is a systemic immunological response (anaphylaxis) or a systemic toxic response (anaphylactoid reaction), the accepted first line of treatment is with epinephrine. In this sense, treatment of an allergic emergency encompasses treatment of anaphylaxis, an anaphylactoid response or both. See Leiberman et al., (2005) J. Allergy Clin. Immunol. 115: S483-S523.

Buccal, Lingual and Sublingual Epinephrine Dosage Forms

The present invention provides methods of treating an allergic emergency, such as anaphylaxis, in a patient, comprising administering to the patient a series of buccal, lingual or sublingual dosage forms comprising epinephrine. The methods described herein can be practiced using any pharmaceutical composition or dosage form containing epinephrine that is appropriate for buccal, lingual or sublingual administration. The discrete dosage forms of the present invention can comprise dosages of from about 1 mg to about 100 mg, and in some embodiments from about 15 mg to about 60 mg, of epinephrine. It is to be understood that epinephrine, as used herein, refers to both the free base form as well as any suitable pharmaceutically acceptable salt of epinephrine including, but not limited to epinephrine bitartrate or epinephrine HCl salt.

In certain embodiments, the methods of the present invention can include the use of a buccal, lingual or sublingual dosage form such as a disintegrating or dissolving tablet (ODTs) formulated for immediate disintegration or dissolution in the patient's mouth. In such embodiments, the buccal, lingual or sublingual tablet can disintegrate or dissolve without extracorporeal water. Thus, the saliva present in the patient's mouth is sufficient to initiate disintegration or dissolution of the sublingual tablet in the oral cavity. In such an embodiment, the epinephrine can be absorbed much more quickly than traditional oral dosage forms and can provide a rapid onset of epinephrine activity via absorption into the systemic circulation.

Ingredients and exemplary buccal, lingual or sublingual formulations can be found in Remington: The Science and Practice of Pharmacy 20th ed. (Lippincott Williams & Wilkins, 2000). The patent literature also contains many disclosures of buccal, lingual and sublingual formulations, including U.S. Pat. Nos. 7,067,116; 7,025,983; 6,923,981; 6,596,298; 6,726,928; 6,709,669; 6,509,040; 6,413,549; 5,976,577; 5,827,541; 5,738,875; 5,648,093; 5,631,023; 5,188,825; 4,020,558; 4,229,447; 3,972,995; 3,870,790; 3,444,858; 2,698,822; 3,632,743, and U.S. Published Application Nos. 20070059361; 20040247648; 20040131661; and 20040028730, each of which is specifically incorporated herein by reference in their entirety. In some embodiments, the dosage forms are prepared using pharmaceutically acceptable excipients. The excipients are known to those skilled in the preparation of buccal, lingual or sublingual dosage forms. For example, excipients that are commonly formulated into buccal, lingual and sublingual dosage forms include maltodextrin, colloidal silicon dioxide, starch, starch syrup, sugar and α-lactose.

In certain embodiments of the buccal, lingual or sublingual dosage forms described herein, excipients acting as disintegrants or dissolution enhancing agents can be incorporated into the formulation to provide for faster tablet disintegration or dissolution. In other embodiments, the buccal, lingual or sublingual epinephrine tablets can be formulated using absorption enhancers to maximize the release rate of the epinephrine into to the systemic circulation. In yet other embodiments, the absorption enhancer is a transmucosal absorption enhancer. Transmucosal absorption enhancers are known in the art and include, but are not limited to, chelators (e.g., EDTA, EGTA), non-ionic surfactants (e.g., 23-lauryl ether, laureth-9, polysorbates (including polysorbate 80), sucrose esters, or dodecylmaltoside), cationic surfactants (e.g., benzalkonium chloride or cetylmethylammonium bromide), anionic surfactants (e.g., sodium dodecyl glycocholate or sodium lauryl sulfate), bile salts and other steroidal detergents (e.g., cholate, deoxycholate, taurocholate, sodium glycocholate, sodium taurocholate, saponins, sodium taurodihydrofusidate or sodium glycodihydrofusidate), fatty acids (e.g., oleic acid, lauric acid capric acid, heptnoic acid, stearic acid, sucrose laurate, isopropyl myristate, sodium myristate or caprylic acid), and non-surfactants (e.g., aprotinin, dextran sulfate, sulfoxides, salicylates, Intravail® or 1-dodecylazacycloheptane-2-one(Azone)), phospholipids (e.g, phosphatidylcholines, lysophosphatidylcholine, or monoooleoyl phosphaltidyl ethanomamine), cyclodextrins, and various alkyl glycosides. See, e.g., Shojaei, 1998, J Pharm Pharmaceut Sci 1:15-30; and Mitra et al., 2002, Encyclopedia of Pharmaceutical Technology pp. 2081-2095. In certain embodiments, the transmucosal absorption enhancer can be Intravail® (Aegis Therapeutics, LLC, San Diego, Calif.). In other embodiments, the transmucosal absorption enhancer can be benzalkonium chloride.

In other embodiments, the active components of the epinephrine dosage forms described herein can further comprise other non-essential or less essential components or excipients known in the art, for example, but by no means limited to diluents, binders, glidants, lubricants, colorants, flavorants, coating materials and the like.

Diluents increase bulk of the composition to facilitate compression of the dosage form. As used herein, diluents include, but are not limited to, compounds such as lactose, starch, mannitol, sorbitol, dextrose, tricalcium phosphate, calcium phosphate; anhydrous lactose, spray-dried lactose; pregelatinized starch, compressible sugar, such as Di-Pac® (Amstar), hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; sodium chloride, and the like.

Binders, as used herein, refer to compounds which impart cohesive qualities to the formulation and include, but are not limited to, compounds such as alginic acid and salts thereof, cellulose derivatives such as carboxymethylcellulose, methylcellulose (e.g., Methocel®), hydroxypropylmethylcellulose, hydroxyethylcellulose, hydroxypropylcellulose (e.g., Klucel®)), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalacian, Veegum®, polyethylene glycol, waxes, sodium alginate, and the like.

Lubricants and glidants are compounds that prevent, reduce or inhibit adhesion or friction of materials. Exemplary lubricants or glidants include, but are not limited to, stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Flavoring agents and/or sweeteners useful in the epinephrine formulations described herein, include, but are not limited to, compounds such as acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

It should be appreciated that there is considerable overlap between additives used in the solid dosage forms described herein. Thus, the above-listed additives should be taken as merely exemplary, and not limiting, of the types of additives that can be included in the buccal, lingual or sublingual dosage forms of the present invention. The amounts of such additives can be readily determined by one skilled in the art, according to the particular properties desired.

Methods of Manufacturing Buccal, Lingual and Sublingual Disintegrating Dosage Forms Comprising Epinephrine

In addition, conventional methods of processing active ingredients and excipients into pharmaceutical compositions and dosage forms for buccal, lingual and sublingual administration are well known to the skilled formulation specialist. For example, various techniques are known in the art that can be used to formulate disintegrating or dissolving sublingual tablet dosage forms. Manufacturing processes for buccal, lingual and sublingual disintegrating tablets are known in the art and include, but are not limited to, conventional tableting techniques, freeze-dried technology, and floss-based tableting technology.

i. Conventional Techniques

Conventional tablet processing features conventional tablet characteristics for ease of handling, packaging, and fast disintegration (T. K. Ghosh, Oct. 29, 2003, American Association of Pharmaceutical Scientists). The technology is based on a combination of physically modified polysaccharides that have water dissolution characteristics that facilitate fast disintegration and high compressibility. The result is a fast-disintegrating tablet that has adequate hardness for packaging in bottles and easy handling.

In certain embodiments, the manufacturing process involves granulating low-moldable sugars (e.g., mannitol, lactose, glucose, sucrose, and erythritol) that show quick dissolution characteristics with high-moldable sugars (e.g., maltose, sorbitol, trehalose, and maltitol). The result is a mixture of excipients that have fast-dissolving and highly moldable characteristics (Hamilton et al., 2005, Drug Deliv. Technol. 5:34-37). The epinephrine can be added, along with other standard tableting excipients, during the granulation or blending processes. The tablets are manufactured at a low compression force followed by an optional humidity conditioning treatment to increase tablet hardness (Parakh et al., 2003, Pharm. Tech. 27:92-100).

In other embodiments, a compressed buccal, lingual or sublingual tablet comprising epinephrine is based on a conventional tableting process involving the direct compression of active ingredients, effervescent excipients, and taste-masking agents (see U.S. Pat. No. 5,223,614, which is herein incorporated by reference in its entirety). The tablet quickly disintegrates because effervescent carbon dioxide is produced upon contact with moisture. The effervescent excipient (known as effervescence couple) is prepared by coating the organic acid crystals using a stoichiometrically lesser amount of base material. The particle size of the organic acid crystals is carefully chosen to be larger than the base excipient to ensure uniform coating of the base excipient onto the acid crystals. The coating process is initiated by the addition of a reaction initiator, which is purified water in this case. The reaction is allowed to proceed only to the extent of completing the base coating on organic acid crystals. The required end-point for reaction termination is determined by measuring carbon dioxide evolution. Then, the excipient is mixed with the active ingredient or active microparticles and with other standard tableting excipients and then compressed into tablets.

In still other embodiments, the buccal, lingual or sublingual tablets are made by combining non-compressible fillers with a taste-masking excipient and active ingredient into a dry blend. The blend is compressed into tablets using a conventional rotary tablet press. Tablets made with this process have higher mechanical strength and are sufficiently robust to be packaged in blister packs or bottles (Aurora et al., 2005, Drug Deliv. Technol. 5:50-54). In other embodiments, the method further incorporates taste-masking sweeteners and flavoring agents such as mint, cherry, and orange. In certain embodiments, epinephrine tablets made with this process should disintegrate in the mouth in 5-45 seconds and can be formulated to be bioequivalent to intramuscular or subcutaneous dosage forms containing epinephrine.

ii. Freeze-Dried Buccal, Lingual or Sublingual Dosage Forms Comprising Epinephrine

The freeze-drying process involves the removal of water (by sublimation upon freeze drying) from the liquid mixture of a drug (e.g., epinephrine), matrix former, and other excipients filled into preformed blister pockets. The formed matrix structure is very porous in nature and rapidly dissolves or disintegrates upon contact with saliva (Sastry et al., 2005, Drug Delivery to the Oral Cavity: Molecule to Market, pp. 311-316).

Common matrix-forming agents include gelatins, dextrans, or alginates which form glassy amorphous mixtures for providing structural strength; saccharides such as mannitol or sorbitol for imparting crystallinity and hardness; and water, which functions as a manufacturing process medium during the freeze-drying step to induce the porous structure upon sublimation. In addition, the matrix may contain taste-masking agents such as sweeteners, flavorants, pH-adjusting agents such as citric acid, and preservatives to ensure the aqueous stability of the suspended drug in media before sublimation.

In this embodiment, Freeze-dried buccal, lingual or sublingual ODTs comprising epinephrine can be manufactured and packaged in polyvinyl chloride or polyvinylidene chloride plastic packs, or they may be packed into laminates or aluminum multilaminate foil pouches to protect the product from external moisture.

Other known methods for manufacturing buccal, lingual or sublingual ODTs include lyophilization (e.g., Lyoc (Farmalyoc, now Cephalon, Franzer, Pa.) and QuickSolv (Janssen Pharmaceutica, Beerse, Belgium). Lyoc is a porous, solid wafer manufactured by lyophilizing an oil-in-water emulsion placed directly in a blister and subsequently sealed. The wafer can accommodate high drug dosing and disintegrates rapidly but has poor mechanical strength (see EP 0159237). QuickSolv tablets are made with a similar technology that creates a porous solid matrix by freezing an aqueous dispersion or solution of the matrix formulation. The process works by removing water using an excess of alcohol (solvent extraction). In certain embodiments, the manufacturing methods which utilize the lyophilization techniques, such as those related to QuickSolv as described above, could be of particular importance for producing buccal, lingual or sublingual ODTs comprising epinephrine. This is especially so in light of the data provided herein which shows the potential negative effect that highly water soluble excipients can have in the absorption of epinephrine in vivo. Thus, a buccal, lingual or sublingual ODT comprising epinephrine manufactured by such a lyophilization technique could provide increased in vivo epinephrine absorption due of the removal of water soluble excipients occurring during the water removal step as described above.

iii. Floss-Based Buccal, Lingual or Sublingual Tablets Comprising Epinephrine

In other embodiments, floss-based tablet technology (e.g., FlashDose, Biovail, Mississauga, ON, Canada) can be used to produce fast-dissolving buccal, lingual or sublingual tablets comprising epinephrine using a floss known as the shearform matrix. This floss is commonly composed of saccharides such as sucrose, dextrose, lactose, and fructose. The saccharides are converted into floss by the simultaneous action of flash-melting and centrifugal force in a heat-processing machine similar to that used to make cotton candy. See U.S. Pat. Nos. 5,587,172, 5,622,717, 5,567,439, 5,871,781, 5,654,003, and 5,622,716, each of which is specifically incorporated by reference herein in their entirety. The fibers produced are usually amorphous in nature and are partially re-crystallized, which results in a free-flowing floss. The floss can be mixed with epinephrine and pharmaceutically acceptable excipients followed by compression into a tablet that has fast-dissolving characteristics.

iv. Additional Methods of Formulating Buccal, Lingual or Sublingual Dosage Forms Comprising Epinephrine

Additional techniques can also be used to formulate the rapidly disintegrating or dissolving buccal, lingual or sublingual dosage forms of the present invention. See, Sastry et al., 2000, Pharm. Sci. Technol. Today 3: 138-145; Chang et al. 2000, Pharm. Technol. 24: 52-58; Sharma et al., 2003 Pharm. Technol. of North America 10-15; and Allen, 2003, Int'l J. of Pharm. Technol. 7: 449-450, each of which is specifically incorporated herein by reference in their entirety. In some embodiments, direct compression can be used to formulate the buccal, lingual or sublingual epinephrine dosage forms of the present invention.

Other techniques useful in formulating the buccal, lingual, or sublingual dosage forms described herein include the formulation of rapidly dissolving oral films. These techniques are known in the art and described in, for example, U.S. Pat. Nos. 7,067,116; 7,025,983; 6,923,981; 6,596,298; and U.S. Published Application No. 20040247648, each of which is specifically incorporated herein in their entirety. In such embodiments, in addition to epinephrine, the rapidly dissolving oral films can comprise a film-forming agent, and at least one of the following additional ingredients: water, antimicrobial agents, plasticizing agents, flavoring agents, saliva stimulating agents, cooling agents, surfactants, stabilizing agents, emulsifying agents, thickening agents, binding agents, coloring agents, sweeteners, fragrances, triglycerides, preservatives, polyethylene oxides, propylene glycol, and the like. By way of a non-limiting example, the buccal, lingual, or sublingual rapidly dissolving oral films described herein can comprise a film-forming agent selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. In certain aspects, the rapidly dissolving films can further comprise a taste-masking agent, e.g., an ion exchange resin. In certain embodiments, the ion exchange resins for use in the dissolving films of the present invention are water-insoluble and consist of a pharmacologically inert organic or inorganic matrix containing covalently bound functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be, e.g., silica gel modified by the addition of ionic groups. The covalently bound ionic groups may be strongly acidic (e.g., sulfonic acid), weakly acidic (e.g., carboxylic acid), strongly basic (e.g., quaternary ammonium), weakly basic (e.g., primary amine), or a combination of acidic and basic groups. In still other aspects, the rapidly dissolving films can comprise modified starches which can significantly improve the overall stability and resistance of the film to adverse factors including heat and moisture for better product performance and improved storage life. Modified starches can also enable the dissolution of more solids (up to twice the amount attainable with unmodified starch) in the buccal, lingual, or sublingual film. In certain embodiments, the modified starches include modified corn starches, modified tapioca starches, acid and enzyme hydrolyzed corn and/or potato starches, hypochlorite-oxidized starches, acid-thinned starches, ethylated starches, cross-bonded starches, hydroxypropylated tapioca starches, hydroxypropylated corn starches, pregelatinized modified starches, and the like.

Still other techniques useful in formulating the buccal, lingual, or sublingual dosage forms described herein include the formulation of rapidly disintegrating or fast dispersing dosage forms which release the epinephrine rapidly on contact with a fluid (e.g., saliva, bodily fluids, water, and the like). These techniques are known in the art and are set forth in, for example, U.S. Pat. Nos. 6,726,928; 6,709,669; 5,976,577; 5,827,541; 5,738,875; 5,631,023; and 5,188,825, each of which is specifically incorporated herein by reference in their entirety. In certain aspects, such methods include the preparation freeze-dried dosage forms comprising epinephrine, wherein the epinephrine is bonded to an ion exchange resin to form a substantially water insoluble complex. This complex is then mixed with a compatible carrier and freeze-dried. In other aspects, such methods include preparation of an oral solid rapidly disintegrating dosage form comprising epinephrine comprising the formation of an aqueous solution and a suspension in an aqueous medium of an uncoated and uncomplexed epinephrine free base together with a carrier material selected from the group consisting of water-soluble and water-dispersible carrier materials and a compound which converts the epinephrine, which is present in its salt form, into its free base form and removing the aqueous medium. In still other aspects, such methods include buccal, lingual or sublingual dosage forms comprising epinephrine further comprising a carrier, wherein the carrier is gelatin and the dosage form is a fast-dispersing dosage form which releases the active ingredient rapidly on contact with a fluid (e.g., saliva or bodily fluids). In certain embodiments, the gelatin is a mammalian-derived gelatin. In other embodiments, the gelatin is a non-mammalian derived gelatin, such as fish gelatin.

Irrespective of the technique used to formulate the buccal, lingual or sublingual epinephrine dosage forms, the methods of the present invention, in certain embodiments, comprise the administration of buccal, lingual or sublingual epinephrine dosage forms comprising an amount of epinephrine having similar bioequivalency to about 0.01 mg/Kg of epinephrine administered by intra-muscular administration. In other embodiments, the buccal, lingual or sublingual dosage forms comprise an amount of epinephrine that is bioequivalent about 0.1 mg to about 0.5 mg of epinephrine administered by intramuscular injection. In one embodiment, the buccal, lingual or sublingual dosage forms comprise an amount of epinephrine that is bioequivalent to about 0.15 mg of epinephrine administered by intra-muscular injection. In another embodiment, the buccal, lingual or sublingual dosage forms comprise an amount of epinephrine that is bioequivalent to about 0.3 mg administered by intramuscular (IM) injection or administration. In one embodiment, the buccal, lingual or sublingual epinephrine dosage forms comprise between about 1 mg to about 100 mg of epinephrine per dosage. In another embodiment, the buccal, lingual or sublingual epinephrine forms comprise between about 15 mg to about 60 mg of epinephrine per dosage. Thus, the methods described herein can provide dosage forms that obviate the need for, and overcome the problems associated with, IM or subcutaneous injections of epinephrine.

In certain embodiments, the methods of the present invention include the use of a buccal, lingual or sublingual dosage form such as a disintegrating or dissolving tablet formulated for immediate disintegration or dissolution in the patient's mouth. In such embodiments, the buccal, lingual or sublingual tablet can disintegrate or dissolve without extracorporeal water. Thus, the saliva present in the patient's mouth is sufficient to initiate disintegration or dissolution of the buccal, lingual or sublingual tablet in the oral cavity. In such an embodiment, the epinephrine can be absorbed much more quickly than traditional oral dosage forms and can provide a rapid onset of epinephrine activity via absorption into the systemic circulation.

Buccal, Lingual or Sublingual Dosing Regimens of Epinephrine for the Treatment of Anaphylaxis

The present invention provides dosing regimens for the treatment of an allergic emergency, such as anaphylaxis in a patient. These dosing regimens provide methods for the treatment of an allergic emergency comprising the steps of (a) administering a first dose of a buccal, lingual or sublingual dosage form comprising epinephrine; (b) administering a second dose of a buccal, lingual or sublingual dosage form comprising epinephrine; (c) optionally administering a third dose of a buccal, lingual or sublingual dosage form comprising epinephrine; (d) optionally administering a fourth dose of a buccal, lingual or sublingual dosage form comprising epinephrine; and (e) optionally administering a fifth dose of a buccal, lingual or sublingual dosage form comprising epinephrine.

In other aspects of the present invention, provided herein are methods for treating an allergic emergency which increase patient compliance with the epinephrine treatment regimen as compared to traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine; (c) optionally administering a third dose of a buccal, lingual or sublingual dosage form comprising epinephrine; (d) optionally administering a fourth dose of a buccal, lingual or sublingual dosage form comprising epinephrine; and (e) optionally administering a fifth dose of a buccal, lingual or sublingual dosage form comprising epinephrine. In certain embodiments, the methods comprise administering a first and second dose. In other embodiments, the methods comprise administering a first, second and third dose. In still other embodiments, the methods comprise administering a first, second, third and fourth dose. In yet other embodiments, the methods comprise administering a first, second, third, fourth and fifth dose.

In still other aspects of the present invention, provided herein are methods for treating an allergic emergency which reduce patient apprehension associated with traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine; (c) optionally administering a third dose of a buccal, lingual or sublingual dosage form comprising epinephrine; (d) optionally administering a fourth dose of a buccal, lingual or sublingual dosage form comprising epinephrine; and (e) optionally administering a fifth dose of a buccal, lingual or sublingual dosage form comprising epinephrine. In certain embodiments, the methods comprise administering a first and second dose. In other embodiments, the methods comprise administering a first, second and third dose. In still other embodiments, the methods comprise administering a first, second, third and fourth dose. In yet other embodiments, the methods comprise administering a first, second, third, fourth and fifth dose.

In yet other aspects of the present invention, provided herein are methods for the treatment of an allergic emergency which eliminate the pain associated with the administration of traditional injectable epinephrine treatment regimens, the methods comprising the steps of (a) administering a first dose of a buccal, lingual or sublingual dosage form comprising epinephrine; (b) administering a second dose of a buccal, lingual or sublingual dosage form comprising epinephrine; (c) optionally administering a third dose of a buccal, lingual or sublingual dosage form comprising epinephrine; (d) optionally administering a fourth dose of a buccal, lingual or sublingual dosage form comprising epinephrine; and (e) optionally administering a fifth dose of a buccal, lingual or sublingual dosage form comprising epinephrine. In certain embodiments, the methods comprise administering a first and second dose. In other embodiments, the methods comprise administering a first, second and third dose. In still other embodiments, the methods comprise administering a first, second, third and fourth dose. In yet other embodiments, the methods comprise administering a first, second, third, fourth and fifth dose.

In certain embodiments, the methods comprise administering a first and second dose. In other embodiments, the methods comprise administering a first, second and third dose. In still other embodiments, the methods comprise administering a first, second, third and fourth dose. In yet other embodiments, the methods comprise administering a first, second, third, fourth and fifth dose. In certain other embodiments, the methods comprise administering more than five doses of epinephrine.

In certain embodiments, the first buccal, lingual or sublingual dosage form comprises an amount of epinephrine that is bioequivalent to about 0.01 mg/Kg of epinephrine administered by intra-muscular administration. In other embodiments, the first buccal, lingual or sublingual dosage form comprises an amount of epinephrine that is bioequivalent to about 0.10 mg to about 0.50 mg of epinephrine administered by intra-muscular injection. In one embodiment, the first buccal, lingual or sublingual dosage form comprises an amount of epinephrine that is bioequivalent to about 0.10 mg of epinephrine administered by intramuscular injection. In another embodiment, the first buccal, lingual or sublingual dosage form comprises an amount of epinephrine that is bioequivalent to about 0.15 mg of epinephrine administered by intra-muscular injection. In still another embodiment, the first buccal, lingual or sublingual dosage form comprises an amount of epinephrine that is bioequivalent to about 0.30 mg of epinephrine administered by intra-muscular injection. In yet another embodiment, the first buccal, lingual or sublingual dosage form comprises an amount of epinephrine that is bioequivalent to about 0.45 mg of epinephrine administered by intra-muscular injection. In still yet another embodiment, the first buccal, lingual or sublingual dosage form comprises an amount of epinephrine that is bioequivalent to about 0.50 mg of epinephrine administered by intramuscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intramuscular administration. In other embodiments, the subsequent administration of a second or greater buccal, lingual, or sublingual dosage form is bioequivalent to the subsequent administration of a second or greater injectable dosage form comprising about 0.10 mg to about 0.50 mg of epinephrine administered by intramuscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intra-muscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intramuscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intramuscular injection.

In certain other embodiments, the dosing regimen comprising the buccal, lingual or sublingual dosage forms comprises an amount of epinephrine that is bioequivalent to about 0.01 mg/Kg of epinephrine administered by intramuscular administration. In other embodiments, the dosing regimen comprising the buccal, lingual or sublingual dosage forms comprises an amount of epinephrine that is bioequivalent to about 0.1 mg to about 0.5 mg of epinephrine administered by intra-muscular injection. In one embodiment, the dosing regimen comprising the buccal, lingual or sublingual dosage forms comprises an amount of epinephrine that is bioequivalent to about 0.10 mg of epinephrine administered by intra-muscular injection. In another embodiment, the dosing regimen comprising the buccal, lingual or sublingual dosage forms comprises an amount of epinephrine that is bioequivalent to about 0.15 mg of epinephrine administered by intramuscular injection. In still another embodiment, the dosing regimen comprising the buccal, lingual or sublingual dosage forms comprises an amount of epinephrine that is bioequivalent to about 0.30 mg of epinephrine administered by intramuscular injection. In yet another embodiment, the dosing regimen comprising the buccal, lingual or sublingual dosage forms comprises an amount of epinephrine that is bioequivalent to about 0.45 mg of epinephrine administered by intramuscular injection. In still yet another embodiment, the dosing regimen comprising the buccal, lingual or sublingual dosage forms comprises an amount of epinephrine that is bioequivalent to about 0.50 mg of epinephrine administered by intramuscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine.

In some embodiments, the buccal, lingual or sublingual dosage forms are tablets or films. In certain embodiments, the dosage form is a lingual tablet or film. In certain other embodiments, the dosage form is a sublingual tablet or film. In other embodiments, the dosage form is a buccal tablet or film. In some embodiments, the buccal, lingual or sublingual dosage forms further comprise a pharmaceutically acceptable excipient.

A. Decreasing Doses of Buccal, Lingual or Sublingual Epinephrine

In other embodiments, decreasing dosing regimens are provided for the treatment of an allergic emergency. These embodiments provide methods comprising the administration of a first dose of a buccal, lingual or sublingual dosage form comprising epinephrine having a higher dosage of epinephrine that the subsequently administered doses of a buccal, lingual or sublingual dosage form comprising epinephrine.

In certain embodiments, the decreasing dose regimens comprise methods which can comprise the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the second dose; (d) optionally administering a fourth dose of a buccal, lingual or sublingual dosage form comprising epinephrine wherein the amount of epinephrine in the fourth dose is about 50% to about 100% the amount of epinephrine in the third dose; and (e) optionally administering a fifth dose of a buccal, lingual or sublingual dosage form comprising epinephrine wherein the amount of epinephrine in the fifth dose is about 50% to about 100% the amount of epinephrine in the fourth dose. In other embodiments, each subsequent dose can be about 10% to about 100% the amount of epinephrine of the prior dose, e.g. the second dose can be about 10% to about 100% the amount of epinephrine of the first dose. In yet other embodiments, each subsequent dose can be about 30% to about 100% the amount of epinephrine of the prior dose. In still other embodiments, each subsequent dose can be about 40% to about 100% the amount of epinephrine of the prior dose.

In certain other embodiments, the decreasing dose regimens comprise methods which can comprise the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 50% to about 100% the amount of epinephrine in the first dose; (c) optionally administering a third dose of a buccal, lingual or sublingual dosage form comprising epinephrine wherein the amount of epinephrine in the third dose is about 50% to about 100% the amount of epinephrine in the first dose; (d) optionally administering a fourth dose of a buccal, lingual or sublingual dosage form comprising epinephrine wherein the amount of epinephrine in the fourth dose is about 50% to about 100% the amount of epinephrine in the first dose; and (e) optionally administering a fifth dose of a buccal, lingual or sublingual dosage form comprising epinephrine wherein the amount of epinephrine in the fifth dose is about 50% to about 100% the amount of epinephrine in the first dose. In other embodiments, each subsequent dose can be about 10% to about 100% the amount of epinephrine of the first dose. In yet other embodiments, each subsequent dose can be about 30% to about 100% the amount of epinephrine of the first dose. In still other embodiments, each subsequent dose can be about 40% to about 100% the amount of epinephrine of the first dose.

In certain embodiments, the first buccal, lingual or sublingual dosage form comprises an amount of epinephrine that is bioequivalent to about 0.01 mg/Kg of epinephrine administered by intra-muscular administration. In other embodiments, the first buccal, lingual or sublingual dosage form comprises an amount of epinephrine that is bioequivalent to about 0.10 mg to about 0.50 mg of epinephrine administered by intramuscular injection. In one embodiment, the first buccal, lingual or sublingual dosage form comprises an amount of epinephrine that is bioequivalent to about 0.10 mg of epinephrine administered by intramuscular injection. In another embodiment, the first buccal, lingual or sublingual dosage form comprises an amount of epinephrine that is bioequivalent to about 0.15 mg of epinephrine administered by intramuscular injection. In still another embodiment, the first buccal, lingual or sublingual dosage form comprises an amount of epinephrine that is bioequivalent to about 0.30 mg of epinephrine administered by intramuscular injection. In yet another embodiment, the first buccal, lingual or sublingual dosage form comprises an amount of epinephrine that is bioequivalent to about 0.45 mg of epinephrine administered by intramuscular injection. In still yet another embodiment, the first buccal, lingual or sublingual dosage form comprises an amount of epinephrine that is bioequivalent to about 0.50 mg of epinephrine administered by intramuscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intramuscular administration. In other embodiments, the subsequent administration of a second or greater buccal, lingual, or sublingual dosage form is bioequivalent to the subsequent administration of a second or greater injectable dosage form comprising about 0.10 mg to about 0.50 mg of epinephrine administered by intramuscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intra-muscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intramuscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intramuscular injection.

In certain other embodiments, the dosing regimen comprising the buccal, lingual or sublingual dosage forms comprises an amount of epinephrine that is bioequivalent to about 0.01 mg/Kg of epinephrine administered by intra-muscular administration. In other embodiments, the dosing regimen comprising the buccal, lingual or sublingual dosage forms comprises an amount of epinephrine that is bioequivalent to about 0.1 mg to about 0.5 mg of epinephrine administered by intramuscular injection. In one embodiment, the dosing regimen comprising the buccal, lingual or sublingual dosage forms comprises an amount of epinephrine that is bioequivalent to about 0.10 mg of epinephrine administered by intra-muscular injection. In another embodiment, the dosing regimen comprising the buccal, lingual or sublingual dosage forms comprises an amount of epinephrine that is bioequivalent to about 0.15 mg of epinephrine administered by intramuscular injection. In still another embodiment, the dosing regimen comprising the buccal, lingual or sublingual dosage forms comprises an amount of epinephrine that is bioequivalent to about 0.30 mg of epinephrine administered by intramuscular injection. In yet another embodiment, the dosing regimen comprising the buccal, lingual or sublingual dosage forms comprises an amount of epinephrine that is bioequivalent to about 0.45 mg of epinephrine administered by intra-muscular injection. In still yet another embodiment, the dosing regimen comprising the buccal, lingual or sublingual dosage forms comprises an amount of epinephrine that is bioequivalent to about 0.50 mg of epinephrine administered by intra-muscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine.

In some embodiments, the buccal, lingual or sublingual dosage forms are tablets or films. In certain embodiments, the dosage form is a lingual tablet or film. In certain other embodiments, the dosage form is a sublingual tablet or film. In other embodiments, the dosage form is a buccal tablet or film. In some embodiments, the buccal, lingual or sublingual dosage forms further comprise a pharmaceutically acceptable excipient.

In certain embodiments, the methods comprise administering a first and second dose of a buccal, lingual or sublingual dosage form comprising epinephrine according to the decreasing dosing regimen described above. In other embodiments, the methods comprise administering a first, second and third dose of a buccal, lingual or sublingual dosage form comprising epinephrine according to the decreasing dosing regimen described above. In still other embodiments, the methods comprise administering a first, second, third and fourth dose of a buccal, lingual or sublingual dosage form comprising epinephrine according to the decreasing dosing regimen described above. In yet other embodiments, the methods comprise administering a first, second, third, fourth and fifth dose of a buccal, lingual or sublingual dosage form comprising epinephrine according to the decreasing dosing regimen described above. In certain other embodiments, the decreasing dosing regimens can comprise methods comprising administering more than five doses of a buccal, lingual or sublingual dosage form comprising epinephrine.

In some aspects of the present invention, the time interval between each consecutive or sequential dose can be the amount of time it takes to see a therapeutic effect in the patient. In some embodiments, the time interval between consecutive or sequential doses ranges from about 3 minutes to about 10 minutes. In other embodiments, the time interval between consecutive or sequential doses is about 5 minutes.

B. Buccal, Lingual or Sublingual Epinephrine Dosing Regimens with Similar Amounts of Epinephrine

In other embodiments, dosing regimens are provided for the treatment of an allergic emergency wherein the multiple doses of a buccal, lingual or sublingual dosage form comprising epinephrine have approximately equal dosages of epinephrine.

In certain embodiments, the dosing regimens described herein comprise methods for treating an allergic emergency which comprise the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the first dose; (d) optionally administering a fourth dose of a buccal, lingual or sublingual dosage form comprising epinephrine wherein the amount of epinephrine in the fourth dose is about 75% to about 125% the amount of epinephrine in the first dose; and (e) optionally administering a fifth dose of a buccal, lingual or sublingual dosage form comprising epinephrine wherein the amount of epinephrine in the fifth dose is about 75% to about 125% the amount of epinephrine in the first dose.

In certain other embodiments, the dosing regimens described herein comprise methods for treating an allergic emergency which comprise the steps of (a) administering a first dose of a buccal, lingual or sublingual dosage form comprising epinephrine; (b) administering a second dose of a buccal, lingual or sublingual dosage form comprising epinephrine wherein the amount of epinephrine in the second dose is about 75% to about 125% the amount of epinephrine in the first dose; (c) optionally administering a third dose of a buccal, lingual or sublingual dosage form comprising epinephrine wherein the amount of epinephrine in the third dose is about 75% to about 125% the amount of epinephrine in the second dose; (d) optionally administering a fourth dose of a buccal, lingual or sublingual dosage form comprising epinephrine wherein the amount of epinephrine in the fourth dose is about 75% to about 125% the amount of epinephrine in the third dose; and (e) optionally administering a fifth dose of a buccal, lingual or sublingual dosage form comprising epinephrine wherein the amount of epinephrine in the fifth dose is about 75% to about 125% the amount of epinephrine in the fourth dose.

In certain embodiments, the first buccal, lingual or sublingual dosage form comprises an amount of epinephrine that is bioequivalent to about 0.01 mg/Kg of epinephrine administered by intramuscular administration. In other embodiments, the first buccal, lingual or sublingual dosage form comprises an amount of epinephrine that is bioequivalent to about 0.10 mg to about 0.50 mg of epinephrine administered by intra-muscular injection. In one embodiment, the first buccal, lingual or sublingual dosage form comprises an amount of epinephrine that is bioequivalent to about 0.10 mg of epinephrine administered by intra-muscular injection. In another embodiment, the first buccal, lingual or sublingual dosage form comprises an amount of epinephrine that is bioequivalent to about 0.15 mg of epinephrine administered by intramuscular injection. In still another embodiment, the first buccal, lingual or sublingual dosage form comprises an amount of epinephrine that is bioequivalent to about 0.30 mg of epinephrine administered by intramuscular injection. In yet another embodiment, the first buccal, lingual or sublingual dosage form comprises an amount of epinephrine that is bioequivalent to about 0.45 mg of epinephrine administered by intramuscular injection. In still yet another embodiment, the first buccal, lingual or sublingual dosage form comprises an amount of epinephrine that is bioequivalent to about 0.50 mg of epinephrine administered by intramuscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intramuscular administration. In other embodiments, the subsequent administration of a second or greater buccal, lingual, or sublingual dosage form is bioequivalent to the subsequent administration of a second or greater injectable dosage form comprising about 0.10 mg to about 0.50 mg of epinephrine administered by intramuscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intramuscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intra-muscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intramuscular injection.

In certain other embodiments, the dosing regimen comprising the buccal, lingual or sublingual dosage forms comprises an amount of epinephrine that is bioequivalent to about 0.01 mg/Kg of epinephrine administered by intramuscular administration. In other embodiments, the dosing regimen comprising the buccal, lingual or sublingual dosage forms comprises an amount of epinephrine that is bioequivalent to about 0.1 mg to about 0.5 mg of epinephrine administered by intramuscular injection. In one embodiment, the dosing regimen comprising the buccal, lingual or sublingual dosage forms comprises an amount of epinephrine that is bioequivalent to about 0.10 mg of epinephrine administered by intramuscular injection. In another embodiment, the dosing regimen comprising the buccal, lingual or sublingual dosage forms comprises an amount of epinephrine that is bioequivalent to about 0.15 mg of epinephrine administered by intra-muscular injection. In still another embodiment, the dosing regimen comprising the buccal, lingual or sublingual dosage forms comprises an amount of epinephrine that is bioequivalent to about 0.30 mg of epinephrine administered by intra-muscular injection. In yet another embodiment, the dosing regimen comprising the buccal, lingual or sublingual dosage forms comprises an amount of epinephrine that is bioequivalent to about 0.45 mg of epinephrine administered by intramuscular injection. In still yet another embodiment, the dosing regimen comprising the buccal, lingual or sublingual dosage forms comprises an amount of epinephrine that is bioequivalent to about 0.50 mg of epinephrine administered by intramuscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine.

In some embodiments, the buccal, lingual or sublingual dosage forms are tablets or films. In certain embodiments, the dosage form is a lingual tablet or film. In certain other embodiments, the dosage form is a sublingual tablet or film. In other embodiments, the dosage form is a buccal tablet or film. In some embodiments, the buccal, lingual or sublingual dosage forms further comprise a pharmaceutically acceptable excipient.

In certain embodiments, the methods comprise administering a first and second dose of a buccal, lingual or sublingual dosage form comprising epinephrine according to the dosing regimen comprising approximately equal dosages of epinephrine described above. In other embodiments, the methods comprise administering a first, second and third dose of a buccal, lingual or sublingual dosage form comprising epinephrine according to the dosing regimen comprising approximately equal dosages of epinephrine described above. In still other embodiments, the methods comprise administering a first, second, third and fourth dose of a buccal, lingual or sublingual dosage form comprising epinephrine according to the dosing regimen comprising approximately equal dosages of epinephrine described above. In yet other embodiments, the methods comprise administering a first, second, third, fourth and fifth dose of a buccal, lingual or sublingual dosage form comprising epinephrine according to the dosing regimen comprising approximately equal dosages of epinephrine described above. In certain other embodiments, the dosing regimen comprising approximately equal dosages of epinephrine can comprise methods comprising administering more than five doses of epinephrine.

In certain embodiments, the methods comprise administering the dosage forms by the patient. In other embodiments, the dosage forms can be administered to the patient by another person, such as a parent, a guardian, a care giver, or a health care professional. In certain embodiments, such healthcare professionals administer in an emergency setting, such as in the field, including ambulances or at a patient's home, etc.

In some aspects of the present invention, the time interval between consecutive or sequential doses can be the amount of time it takes to see a therapeutic effect in the patient. In other embodiments, the time interval between consecutive or sequential doses ranges from about 3 minutes to about 10 minutes. In one embodiment, the time interval between consecutive or sequential doses is about 5 minutes.

C. Dosage Regimens Comprising Buccal, Lingual or Sublingual Epinephrine in Combination with an Absorption Enhancer

The term “absorption enhancer,” as used herein, refers to a chemical agent that when present in a buccal, lingual or sublingual epinephrine dosage form, increases the absorption of the epinephrine from the buccal, lingual or sublingual dosage form into the systemic circulation of a patient as compared to a buccal, lingual or sublingual epinephrine dosage form not comprising an absorption enhancer. In some embodiments, the methods described herein provide for the use of a buccal, lingual or sublingual dosage form further comprising an absorption enhancer. In certain embodiments, the absorption enhancer can be a transmucosal absorption enhancer. Transmucosal absorption enhancers are known in the art and include, but are not limited to, chelators (e.g., EDTA, EGTA), non-ionic surfactants (e.g., 23-lauryl ether, laureth-9, polysorbates (including polysorbate 80), sucrose esters, or dodecylmaltoside), cationic surfactants (e.g., benzalkonium chloride or cetylmethylammonium bromide), anionic surfactants (e.g., sodium dodecyl glycocholate or sodium lauryl sulfate), bile salts and other steroidal detergents (e.g., cholate, deoxycholate, taurocholate, sodium glycocholate, sodium taurocholate, saponins, sodium taurodihydrofusidate or sodium glycodihydrofusidate), fatty acids (e.g., oleic acid, lauric acid capric acid, heptnoic acid, stearic acid, sucrose laurate, isopropyl myristate, sodium myristate or caprylic acid), and non-surfactants (e.g., aprotinin, dextran sulfate, sulfoxides, salicylates, Intravail® or 1-dodecylazacycloheptane-2-one(Azone)), phospholipids (e.g, phosphatidylcholines, lysophosphatidylcholine, or monoooleoyl phosphaltidyl ethanomamine), cyclodextrins, and various alkyl glycosides. See, e.g., Shojaei, 1998, J Pharm Pharmaceut Sci 1:15-30; and Mitra et al., 2002, Encyclopedia of Pharmaceutical Technology pp. 2081-2095. In certain embodiments, the transmucosal absorption enhancer useful in the methods described herein is Intravail® (Aegis Therapeutics, LLC, San Diego, Calif.). In other embodiments, the transmucosal absorption enhancer useful in the methods described herein is benzalkonium chloride.
i. Decreasing Dosage Regimens of Buccal, Lingual or Sublingual Epinephrine Dosage Forms Comprising an Absorption Enhancer

In some embodiments, the methods of the present invention can comprise the steps of (a) administering a first dose of a buccal, lingual or sublingual dosage form comprising epinephrine and at least one absorption enhancer; (b) administering a second dose of a buccal, lingual or sublingual dosage form comprising epinephrine and at least one absorption enhancer wherein the amount of epinephrine in the second dose is about 50% to about 100% the amount of epinephrine in the first dose; (c) optionally administering a third dose of a buccal, lingual or sublingual dosage form comprising epinephrine and at least one absorption enhancer wherein the amount of epinephrine in the third dose is about 50% to about 100% the amount of epinephrine in the second dose; (d) optionally administering a fourth dose of a buccal, lingual or sublingual dosage form comprising epinephrine and at least one absorption enhancer wherein the amount of epinephrine in the fourth dose is about 50% to about 100% the amount of epinephrine in the third dose; and (e) optionally administering a fifth dose of a buccal, lingual or sublingual dosage form comprising epinephrine and at least one absorption enhancer wherein the amount of epinephrine in the fifth dose is about 50% to about 100% the amount of epinephrine in the fourth dose.

In other embodiments, the methods of the present invention can comprise the steps of (a) administering a first dose of a buccal, lingual or sublingual dosage form comprising epinephrine and at least one absorption enhancer; (b) administering a second dose of a buccal, lingual or sublingual dosage form comprising epinephrine and at least one absorption enhancer wherein the amount of epinephrine in the second dose is about 50% to about 100% the amount of epinephrine in the first dose; (c) optionally administering a third dose of a buccal, lingual or sublingual dosage form comprising epinephrine and at least one absorption enhancer wherein the amount of epinephrine in the third dose is about 50% to about 100% the amount of epinephrine in the first dose; (d) optionally administering a fourth dose of a buccal, lingual or sublingual dosage form comprising epinephrine and at least one absorption enhancer wherein the amount of epinephrine in the fourth dose is about 50% to about 100% the amount of epinephrine in the first dose; and (e) optionally administering a fifth dose of a buccal, lingual or sublingual dosage form comprising epinephrine and at least one absorption enhancer wherein the amount of epinephrine in the fifth dose is about 50% to about 100% the amount of epinephrine in the first dose.

In certain embodiments, the first buccal, lingual or sublingual dosage form comprises an amount of epinephrine that is bioequivalent to about 0.01 mg/Kg of epinephrine administered by intramuscular administration. In other embodiments, the first buccal, lingual or sublingual dosage form comprises an amount of epinephrine that is bioequivalent to about 0.10 mg to about 0.50 mg of epinephrine administered by intramuscular injection. In one embodiment, the first buccal, lingual or sublingual dosage form comprises an amount of epinephrine that is bioequivalent to about 0.10 mg of epinephrine administered by intra-muscular injection. In another embodiment, the first buccal, lingual or sublingual dosage form comprises an amount of epinephrine that is bioequivalent to about 0.15 mg of epinephrine administered by intra-muscular injection. In still another embodiment, the first buccal, lingual or sublingual dosage form comprises an amount of epinephrine that is bioequivalent to about 0.30 mg of epinephrine administered by intramuscular injection. In yet another embodiment, the first buccal, lingual or sublingual dosage form comprises an amount of epinephrine that is bioequivalent to about 0.45 mg of epinephrine administered by intra-muscular injection. In still yet another embodiment, the first buccal, lingual or sublingual dosage form comprises an amount of epinephrine that is bioequivalent to about 0.50 mg of epinephrine administered by intra-muscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intramuscular administration. In other embodiments, the subsequent administration of a second or greater buccal, lingual, or sublingual dosage form is bioequivalent to the subsequent administration of a second or greater injectable dosage form comprising about 0.10 mg to about 0.50 mg of epinephrine administered by intramuscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intramuscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intra-muscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intramuscular injection.

In certain other embodiments, the dosing regimen comprising the buccal, lingual or sublingual dosage forms comprises an amount of epinephrine that is bioequivalent to about 0.01 mg/Kg of epinephrine administered by intramuscular administration. In other embodiments, the dosing regimen comprising the buccal, lingual or sublingual dosage forms comprises an amount of epinephrine that is bioequivalent to about 0.1 mg to about 0.5 mg of epinephrine administered by intramuscular injection. In one embodiment, the dosing regimen comprising the buccal, lingual or sublingual dosage forms comprises an amount of epinephrine that is bioequivalent to about 0.10 mg of epinephrine administered by intra-muscular injection. In another embodiment, the dosing regimen comprising the buccal, lingual or sublingual dosage forms comprises an amount of epinephrine that is bioequivalent to about 0.15 mg of epinephrine administered by intramuscular injection. In still another embodiment, the dosing regimen comprising the buccal, lingual or sublingual dosage forms comprises an amount of epinephrine that is bioequivalent to about 0.30 mg of epinephrine administered by intramuscular injection. In yet another embodiment, the dosing regimen comprising the buccal, lingual or sublingual dosage forms comprises an amount of epinephrine that is bioequivalent to about 0.45 mg of epinephrine administered by intramuscular injection. In still yet another embodiment, the dosing regimen comprising the buccal, lingual or sublingual dosage forms comprises an amount of epinephrine that is bioequivalent to about 0.50 mg of epinephrine administered by intramuscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine.

In certain embodiments, the methods comprise administering a first and second dose of a buccal, lingual or sublingual dosage form comprising epinephrine according to the decreasing dosing regimen described above. In other embodiments, the methods comprise administering a first, second and third dose of a buccal, lingual or sublingual dosage form comprising epinephrine according to the decreasing dosing regimen described above. In still other embodiments, the methods comprise administering a first, second, third and fourth dose of a buccal, lingual or sublingual dosage form comprising epinephrine according to the decreasing dosing regimen described above. In yet other embodiments, the methods comprise administering a first, second, third, fourth and fifth dose of a buccal, lingual or sublingual dosage form comprising epinephrine according to the decreasing dosing regimen described above. In certain other embodiments, the decreasing dosing regimen can comprise methods comprising administering more than five doses of epinephrine.

In some aspects of the present invention, the time interval between each consecutive or sequential dose can be the amount of time it takes to see a therapeutic effect in the patient. In some embodiments, the time interval between consecutive or sequential doses ranges from about 3 minutes to about 10 minutes. In other embodiments, the time interval between consecutive or sequential doses is about 5 minutes.

It is hypothesized that in some instances, the administration of multiple, consecutive doses of epinephrine can result in an earlier administered epinephrine dose auto-suppressing the absorption of a later administered epinephrine dose. In such cases, the subsequent doses of epinephrine could have lower absorption rates than the earlier administered doses of epinephrine. To overcome this hypothesized effect, the methods of the present invention can comprise dosage regimens wherein the first dose does not comprise an absorption enhancer and the subsequent doses do contain an absorption enhancer. In such embodiments, the absorption enhancer can be present in only the second of two doses. In other embodiments, the absorption enhancer can be present in only the second and third of three doses. In still other embodiments, the absorption enhancer can be present in only the second, third and fourth of four doses. In yet other embodiments, the absorption enhancer can be present in only the second, third, fourth, and fifth of five doses.

In some aspects of the present invention, the time interval between consecutive or sequential doses can be the amount of time it takes to see a therapeutic effect in the patient. In other embodiments, the time interval between consecutive or sequential doses ranges from about 3 minutes to about 10 minutes. In one embodiment, the time interval between consecutive or sequential doses is about 5 minutes.
ii. Similar Dosage Regimens of Buccal, Lingual or Sublingual Epinephrine Dosage Forms Comprising Absorption Enhancers

In addition, the methods of the present invention can comprise the steps of (a) administering a first dose of a buccal, lingual or sublingual dosage form comprising epinephrine and at least one absorption enhancer; (b) administering a second dose of a buccal, lingual or sublingual dosage form comprising epinephrine and at least one absorption enhancer wherein the amount of epinephrine in the second dose is about 75% to about 125% the amount of epinephrine in the first dose; (c) optionally administering a third dose of a buccal, lingual or sublingual dosage form comprising epinephrine and at least one absorption enhancer wherein the amount of epinephrine in the third dose is about 75% to about 125% the amount of epinephrine in the first dose; (d) optionally administering a fourth dose of a buccal, lingual or sublingual dosage form comprising epinephrine and at least one absorption enhancer wherein the amount of epinephrine in the fourth dose is about 75% to about 125% the amount of epinephrine in the first dose; and (e) optionally administering a fifth dose of a buccal, lingual or sublingual dosage form comprising epinephrine and at least one absorption enhancer wherein the amount of epinephrine in the fifth dose is about 75% to about 125% the amount of epinephrine in the first dose.

In addition, the methods of the present invention can comprise the steps of (a) administering a first dose of a buccal, lingual or sublingual dosage form comprising epinephrine and at least one absorption enhancer; (b) administering a second dose of a buccal, lingual or sublingual dosage form comprising epinephrine and at least one absorption enhancer wherein the amount of epinephrine in the second dose is about 75% to about 125% the amount of epinephrine in the first dose; (c) optionally administering a third dose of a buccal, lingual or sublingual dosage form comprising epinephrine and at least one absorption enhancer wherein the amount of epinephrine in the third dose is about 75% to about 125% the amount of epinephrine in the second dose; (d) optionally administering a fourth dose of a buccal, lingual or sublingual dosage form comprising epinephrine and at least one absorption enhancer wherein the amount of epinephrine in the fourth dose is about 75% to about 125% the amount of epinephrine in the third dose; and (e) optionally administering a fifth dose of a buccal, lingual or sublingual dosage form comprising epinephrine and at least one absorption enhancer wherein the amount of epinephrine in the fifth dose is about 75% to about 125% the amount of epinephrine in the fourth dose.

In certain embodiments, the first buccal, lingual or sublingual dosage form comprises an amount of epinephrine that is bioequivalent to about 0.01 mg/Kg of epinephrine administered by intra-muscular administration. In other embodiments, the first buccal, lingual or sublingual dosage form comprises an amount of epinephrine that is bioequivalent to about 0.10 mg to about 0.50 mg of epinephrine administered by intramuscular injection. In one embodiment, the first buccal, lingual or sublingual dosage form comprises an amount of epinephrine that is bioequivalent to about 0.10 mg of epinephrine administered by intra-muscular injection. In another embodiment, the first buccal, lingual or sublingual dosage form comprises an amount of epinephrine that is bioequivalent to about 0.15 mg of epinephrine administered by intramuscular injection. In still another embodiment, the first buccal, lingual or sublingual dosage form comprises an amount of epinephrine that is bioequivalent to about 0.30 mg of epinephrine administered by intra-muscular injection. In yet another embodiment, the first buccal, lingual or sublingual dosage form comprises an amount of epinephrine that is bioequivalent to about 0.45 mg of epinephrine administered by intra-muscular injection. In still yet another embodiment, the first buccal, lingual or sublingual dosage form comprises an amount of epinephrine that is bioequivalent to about 0.50 mg of epinephrine administered by intra-muscular injection. In yet other embodiments, the first buccal, lingual or sublingual dosage form comprises from about 1 mg to about 100 mg of epinephrine. In still other embodiments, the first buccal, lingual or sublingual dosage form comprises from about 15 mg to about 60 mg of epinephrine.

In other embodiments of the present invention, the subsequent administration of a second or greater buccal, lingual, or sublingual dosage form is bioequivalent to the subsequent administration of a second or greater injectable dosage form. In certain embodiments, the subsequent administration of a second or greater buccal, lingual, or sublingual dosage form is bioequivalent to the subsequent administration of a second or greater injectable dosage form comprising about 0.01 mg/Kg of epinephrine administered by intramuscular administration. In other embodiments, the subsequent administration of a second or greater buccal, lingual, or sublingual dosage form is bioequivalent to the subsequent administration of a second or greater injectable dosage form comprising about 0.10 mg to about 0.50 mg of epinephrine administered by intra-muscular injection. In still other embodiments, the subsequent administration of a second or greater buccal, lingual, or sublingual dosage form is bioequivalent to the subsequent administration of a second or greater injectable dosage form comprising about 0.15 mg epinephrine administered by intra-muscular injection. In yet other embodiments, the subsequent administration of a second or greater buccal, lingual, or sublingual dosage form is bioequivalent to the subsequent administration of a second or greater injectable dosage form comprising about 0.30 epinephrine administered by intra-muscular injection. In yet still other embodiments, the subsequent administration of a second or greater buccal, lingual, or sublingual dosage form is bioequivalent to the subsequent administration of a second or greater injectable dosage form comprising about 0.50 mg of epinephrine administered by intra-muscular injection.

In certain other embodiments, the dosing regimen comprising the buccal, lingual or sublingual dosage forms comprises an amount of epinephrine that is bioequivalent to about 0.01 mg/Kg of epinephrine administered by intramuscular administration. In other embodiments, the dosing regimen comprising the buccal, lingual or sublingual dosage forms comprises an amount of epinephrine that is bioequivalent to about 0.1 mg to about 0.5 mg of epinephrine administered by intramuscular injection. In one embodiment, the dosing regimen comprising the buccal, lingual or sublingual dosage forms comprises an amount of epinephrine that is bioequivalent to about 0.10 mg of epinephrine administered by intra-muscular injection. In another embodiment, the dosing regimen comprising the buccal, lingual or sublingual dosage forms comprises an amount of epinephrine that is bioequivalent to about 0.15 mg of epinephrine administered by intra-muscular injection. In still another embodiment, the dosing regimen comprising the buccal, lingual or sublingual dosage forms comprises an amount of epinephrine that is bioequivalent to about 0.30 mg of epinephrine administered by intra-muscular injection. In yet another embodiment, the dosing regimen comprising the buccal, lingual or sublingual dosage forms comprises an amount of epinephrine that is bioequivalent to about 0.45 mg of epinephrine administered by intramuscular injection. In still yet another embodiment, the dosing regimen comprising the buccal, lingual or sublingual dosage forms comprises an amount of epinephrine that is bioequivalent to about 0.50 mg of epinephrine administered by intra-muscular injection. In yet other embodiments, the dosing regimen comprising the buccal, lingual or sublingual dosage forms comprises from about 1 mg to about 100 mg of epinephrine. In still other embodiments, the dosing regimen comprising the buccal, lingual or sublingual dosage forms comprises from about 15 mg to about 60 mg of epinephrine.

In certain embodiments, the methods comprise administering a first and second dose of a buccal, lingual or sublingual dosage form comprising epinephrine according to the dosing regimen comprising approximately equal dosages of epinephrine described above. In other embodiments, the methods comprise administering a first, second and third dose of a buccal, lingual or sublingual dosage form comprising epinephrine according to the dosing regimen comprising approximately equal dosages of epinephrine described above. In still other embodiments, the methods comprise administering a first, second, third and fourth dose of a buccal, lingual or sublingual dosage form comprising epinephrine according to the dosing regimen comprising approximately equal dosages of epinephrine described above. In yet other embodiments, the methods comprise administering a first, second, third, fourth and fifth dose of a buccal, lingual or sublingual dosage form comprising epinephrine according to the dosing regimen comprising approximately equal dosages of epinephrine describe above. In certain other embodiments, the dosing regimen comprising approximately equal dosages of epinephrine can comprise methods comprising administering more than five doses of epinephrine.

In some aspects of the present invention, the time interval between each consecutive or sequential dose can be the amount of time it takes to see a therapeutic effect in the patient. In some embodiments, the time interval between consecutive or sequential doses ranges from about 3 minutes to about 10 minutes. In other embodiments, the time interval between consecutive or sequential doses is about 5 minutes.

As stated above, it is hypothesized that the administration of multiple, consecutive doses of epinephrine can result in an earlier administered epinephrine dose auto-suppressing the absorption of a later administered epinephrine dose. In such cases, the subsequent doses of epinephrine could have lower absorption rates than the earlier administered doses of epinephrine. To overcome this hypothesized effect, the methods of the present invention can comprise dosage regimens wherein the first dose does not comprise an absorption enhancer and the subsequent doses do contain an absorption enhancer. In such embodiments, the absorption enhancer can be present in only the second of two doses. In other embodiments, the absorption enhancer can be present in only the second and third of three doses. In still other embodiments, the absorption enhancer can be present in only the second, third and fourth of four doses. In yet other embodiments, the absorption enhancer can be present in only the second, third, fourth, and fifth of five doses.

In some aspects of the present invention, the time interval between consecutive or sequential doses can be the amount of time it takes to see a therapeutic effect in the patient. In other embodiments, the time interval between consecutive or sequential doses ranges from about 3 minutes to about 10 minutes. In one embodiment, the time interval between consecutive or sequential doses is about 5 minutes.

Rectal Dosing Regimens of Epinephrine for the Treatment of Anaphylaxis

The present invention further provides rectal dosing regimens for the treatment of an allergic emergency, such as anaphylaxis in a patient. These rectal dosing regimens provide methods for the treatment of an allergic emergency comprising the steps of (a) administering a first dose of a rectal dosage form comprising epinephrine; (b) administering a second dose of a rectal dosage form comprising epinephrine; (c) optionally administering a third dose of a rectal dosage form comprising epinephrine; (d) optionally administering a fourth dose of a rectal dosage form comprising epinephrine; and (e) optionally administering a fifth dose of a rectal dosage form comprising epinephrine.

In certain embodiments, the methods comprise administering a first and second dose according to the rectal dosing regimen described above. In other embodiments, the methods comprise administering a first, second and third dose according to the rectal dosing regimen described above. In still other embodiments, the methods comprise administering a first, second, third and fourth dose according to the rectal dosing regimen described above. In yet other embodiments, the methods comprise administering a first, second, third, fourth and fifth dose according to the rectal dosing regimen described above. In certain other embodiments, the rectal dosing regimen comprises methods comprising administering more than five doses of epinephrine.

In certain embodiments, the first rectal dosage form comprises an amount of epinephrine that is bioequivalent to about 0.01 mg/Kg of epinephrine administered by intramuscular administration. In other embodiments, the first rectal dosage form comprises an amount of epinephrine that is bioequivalent to about 0.10 mg to about 0.50 mg of epinephrine administered by intra-muscular injection. In one embodiment, the first rectal dosage form comprises an amount of epinephrine that is bioequivalent to about 0.10 mg of epinephrine administered by intramuscular injection. In another embodiment, the first rectal dosage form comprises an amount of epinephrine that is bioequivalent to about 0.15 mg of epinephrine administered by intramuscular injection. In still another embodiment, the first rectal dosage form comprises an amount of epinephrine that is bioequivalent to about 0.30 mg of epinephrine administered by intra-muscular injection. In yet another embodiment, the first rectal dosage form comprises an amount of epinephrine that is bioequivalent to about 0.45 mg of epinephrine administered by intramuscular injection. In still yet another embodiment, the first rectal dosage form comprises an amount of epinephrine that is bioequivalent to about 0.50 mg of epinephrine administered by intramuscular injection. In yet other embodiments, the first rectal dosage form comprises from about 1 mg to about 100 mg of epinephrine. In still other embodiments, the first rectal dosage form comprises from about 15 mg to about 60 mg of epinephrine.

In other embodiments of the present invention, the subsequent administration of a second or greater rectal dosage form is bioequivalent to the subsequent administration of a second or greater injectable dosage form. In certain embodiments, the subsequent administration of a second or greater rectal dosage form is bioequivalent to the subsequent administration of a second or greater injectable dosage form comprising about 0.01 mg/Kg of epinephrine administered by intra-muscular administration. In other embodiments, the subsequent administration of a second or greater rectal dosage form is bioequivalent to the subsequent administration of a second or greater injectable dosage form comprising about 0.1 mg to about 0.5 mg of epinephrine administered by intra-muscular injection.

In certain other embodiments, the dosing regimen comprising the rectal dosage forms comprises an amount of epinephrine that is bioequivalent to about 0.01 mg/Kg of epinephrine administered by intramuscular administration. In other embodiments, the dosing regimen comprising the rectal dosage forms comprises an amount of epinephrine that is bioequivalent to about 0.1 mg to about 0.5 mg of epinephrine administered by intra-muscular injection. In one embodiment, the dosing regimen comprising the rectal dosage forms comprises an amount of epinephrine that is bioequivalent to about 0.10 mg of epinephrine administered by intramuscular injection. In another embodiment, the dosing regimen comprising the rectal dosage forms comprises an amount of epinephrine that is bioequivalent to about 0.15 mg of epinephrine administered by intramuscular injection. In still another embodiment, the dosing regimen comprising the rectal dosage forms comprises an amount of epinephrine that is bioequivalent to about 0.30 mg of epinephrine administered by intramuscular injection. In yet another embodiment, the dosing regimen comprising the rectal dosage forms comprises an amount of epinephrine that is bioequivalent to about 0.45 mg of epinephrine administered by intra-muscular injection. In still yet another embodiment, the dosing regimen comprising the rectal dosage forms comprises an amount of epinephrine that is bioequivalent to about 0.50 mg of epinephrine administered by intra-muscular injection. In yet other embodiments, the dosing regimen comprising the rectal dosage forms comprises from about 1 mg to about 100 mg of epinephrine. In still other embodiments, the dosing regimen comprising the rectal dosage forms comprises from about 15 mg to about 60 mg of epinephrine.

The rectal dosage forms useful for the methods described herein include, but are not limited to, suppositories, rectal capsules, gels, creams, and ointments. In certain embodiments, the rectal dosage forms further comprise a pharmaceutically acceptable excipient. In one embodiment, the rectal dosage form is a suppository comprising epinephrine and a pharmaceutically acceptable excipient.

In some embodiments, the time interval between each consecutive or sequential rectal dose can be the amount of time it takes to see a therapeutic effect in the patient. In some embodiments, the time interval between consecutive or sequential rectal doses ranges from about 3 minutes to about 10 minutes. In other embodiments, the time interval between consecutive or sequential rectal doses is about 5 minutes.

Kits and Packaging System Comprising Doses of Buccal, Lingual or Sublingual Epinephrine

The present invention is further directed to a kit or packaging system for administration of multiple doses of epinephrine in a buccal, lingual or sublingual dosage form to a patient in need thereof, such as a patient experiencing anaphylaxis, an anaphylactoid reaction or a set of symptoms resembling anaphylaxis or anaphylactoid reaction of unknown etiology but suspected of being an allergic emergency. The kit or packaging system can comprise two or more buccal, lingual or sublingual doses of epinephrine according to the methods described herein. In certain embodiments, the kits or packaging system can further comprise such additional matter as may be necessary to ease administration of the epinephrine to the patient.

In some aspects of the present invention, the kits or packaging systems described herein can comprise two or more doses of buccal, lingual or sublingual epinephrine dosage forms wherein the second or more subsequent doses of epinephrine comprise a dosage of epinephrine that is less than or equal to the dosage of epinephrine in the first dose. In one embodiment, the kits or packaging system can comprise two or more doses of buccal, lingual or sublingual epinephrine dosage forms wherein the second or more subsequent doses of epinephrine comprise a dosage of epinephrine that is about 50% to about 100% the amount of epinephrine in the first dose (Dose 1). In another embodiment, the kits or packaging system can comprise two or more doses of buccal, lingual or sublingual epinephrine dosage forms wherein the second or more subsequent doses of epinephrine comprise a dosage of epinephrine that is about 50% to about 100% the amount of epinephrine in the preceding dose. In other aspects of the present invention, the kits or packaging systems described herein can comprise two or more doses of buccal, lingual or sublingual epinephrine dosage forms wherein the second or more subsequent doses of epinephrine comprise a dosage of epinephrine that ranges from about 75% to about 125% the dosage of the first dose (Dose 1). In still other embodiments, the kits or packaging systems described herein can comprise two or more doses of buccal, lingual or sublingual epinephrine tablets. In yet other embodiments, the kits or packaging systems described herein can comprise two or more doses of buccal, lingual or sublingual epinephrine tablets further comprising a pharmaceutically acceptable excipient.

In some embodiments, the kit or packaging system can comprises two or more doses of a buccal, lingual or sublingual dosage form comprising epinephrine contained within protective packaging which prevents damage due to moisture, light or exposure to oxygen. In one embodiment, the protective packaging comprises a polymer-lined foil. In another embodiment, the protective packaging comprises a blister package. In certain embodiments, the protective packaging comprises a blister package comprises a blister package wherein each individual a buccal, lingual or sublingual dosage form comprising epinephrine is contained within an individual blister.

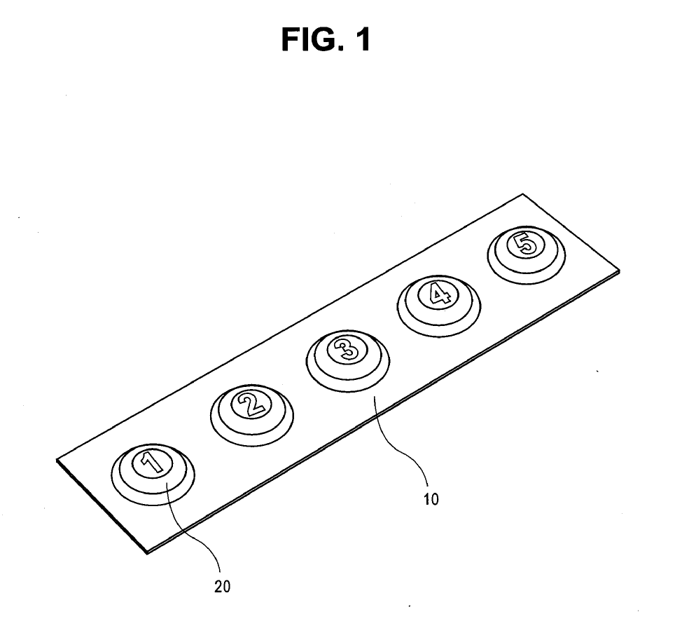

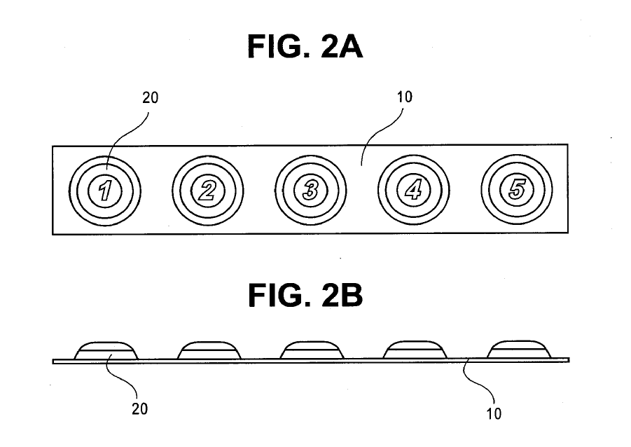

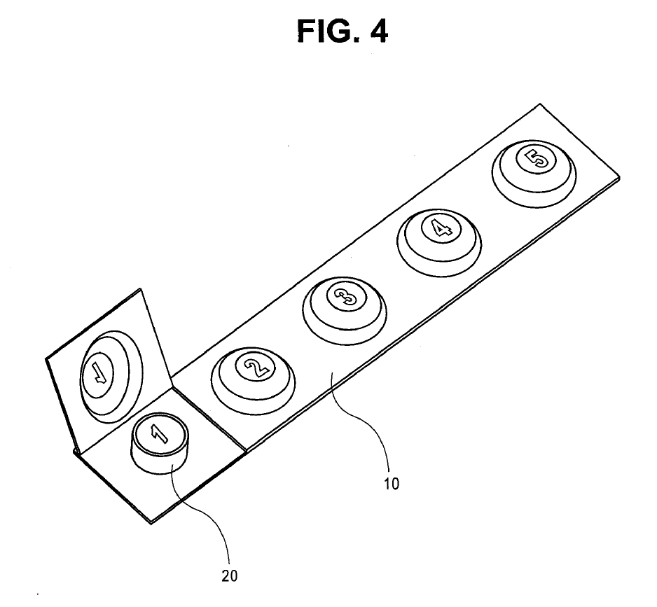

In other embodiments, the kit or packaging system can further comprise a packaging scheme wherein the buccal, lingual or sublingual epinephrine dosage forms are contained within a protective packaging wherein the doses are identified as the first, second, third, fourth and fifth dose, etc. (or first to fourth, or first to third, or first and second, etc., depending on the number of total doses), either by sequential location of the doses within the foil or by the appropriate markings on the dosage forms. A packaging system according to one embodiment of the invention is illustrated in FIG. 4, wherein the packaging system comprises five (5) dosage forms having the numerical markings 1-5 etched on the dosage forms. In certain other embodiments, the kit or packaging system can further comprise a packaging scheme wherein the buccal, lingual or sublingual epinephrine dosage forms are identified by the shape of the dosage form, by the color of the dosage form, by the size of the dosage form, or by a numerical marking embossed on the packaging. A packaging system according to another embodiment is illustrated in FIG. 1 and FIG. 2(a) and 2(b), wherein the packaging system comprises five (5) dosage forms identified by the numerical markings 1-5 embossed on the packaging.

In another embodiment, the kit or packaging system can further comprise directions or instructions for administration of the multiple buccal, lingual or sublingual epinephrine dosage forms. In such an embodiment, the directions or instructions for administration can provide information regarding the sequence in which the buccal, lingual or sublingual epinephrine dosage forms are to be administered. In another such embodiment, the directions or instructions for administration can provide information regarding the timing interval for administration of the buccal, lingual or sublingual epinephrine dosage forms.

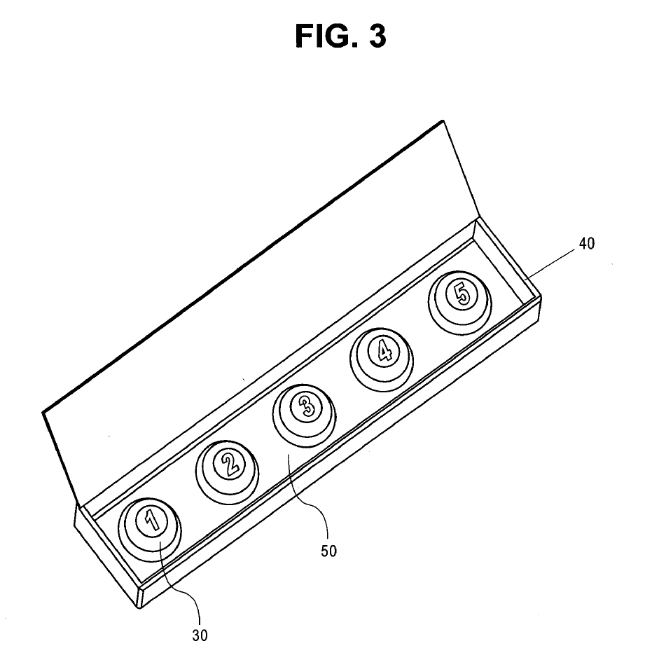

In another embodiment, the kit or packaging system can further comprise a carrying case into which the protective packaging can be placed for convenient storage. For example, a packaging system comprising a carrying case is illustrated in FIG. 3, wherein the packaging system comprises five (5) dosage forms identified by the numerical markings 1-5 embossed on the packaging and the five dosage forms are housed within a protective carrying case for easy portability.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following ingredients, processes and procedures for practicing the methods described herein correspond to that described above. The procedures below describe specific embodiments of methods of administering buccal, lingual or sublingual dosage forms as described herein. Any methods or materials not particularly described in the following examples are within the scope of the invention and will be apparent to those skilled in the art with reference to the disclosure herein.

### Example 1

Administration of a Buccal Dosage Form Comprising Epinephrine for the Treatment of Anaphylaxis

A patient experiencing an allergic emergency initiates treatment at the onset of shortness of breath by self administering 40 mg of epinephrine free base in a buccal dosage form. After approximately 5 minutes pass without amelioration of the symptoms of anaphylaxis, the patient self administers a second 40 mg of epinephrine free base in a buccal dosage form. Within about five minutes after the administration of the second 40 mg of epinephrine free base in a buccal dosage form, the patient's symptoms of anaphylaxis are relieved.

### Example 2

Administration of a Buccal Dosage Form Comprising Epinephrine for the Treatment of Anaphylaxis

A patient experiencing an allergic emergency initiates treatment at the onset of shortness of breath by self administering 40 mg of epinephrine free base in a buccal dosage form. After approximately 5 minutes pass without amelioration of the symptoms of anaphylaxis, the patient self administers a second buccal dosage form comprising 40 mg of epinephrine free base. After another approximately 5 minutes pass without amelioration of the symptoms of anaphylaxis, the patient self administers a third buccal dosage form comprising 20 mg of epinephrine free base. Within about five minutes after the administration of the third buccal epinephrine dosage form, the patient's symptoms of anaphylaxis are relieved.

### Example 3

Administration of a Lingual Dosage Form Comprising Epinephrine for the Treatment of Anaphylaxis

A patient experiencing an allergic emergency initiates treatment at the onset of shortness of breath by self administering 40 mg of epinephrine free base in a lingual dosage form. After approximately 5 minutes pass without amelioration of the symptoms of anaphylaxis, the patient self administers a second lingual dosage form comprising 40 mg of epinephrine free base. After another approximately 5 minutes pass without amelioration of the symptoms of anaphylaxis, the patient self administers a third lingual dosage form comprising 20 mg of epinephrine free base. Within about five minutes after the administration of the third lingual epinephrine dosage form, the patient's symptoms of anaphylaxis are relieved.

### Example 4

A Kit Comprising Multiple Sublingual Dosage Forms of Epinephrine for the Treatment of Anaphylaxis

A kit is provided which contains three sublingual dosages forms each containing 40 mg of epinephrine. The sublingual dosage forms of the kit are packaged in a foil blister pack with numerical markings identifying the order and location of each dose.

Specifically, the foil blister pack containing the three dosage forms is embossed with the numbers 1, 2, and 3, respectively. The numerical markings provide easy identification of each dosage form by the patient. The kit further contains written instructions to aid the patient in administering the dosage forms of epinephrine contained therein in the correct order and at the correct time.

The instructions provide as follows: (a) the first sublingual dosage form, labeled as 1, is to be placed under the tongue of the patient as soon as the patient begins experiencing symptoms of anaphylaxis and maintained there until fully dissolved; (b) if the symptoms of anaphylaxis do not improve or terminate within approximately five minutes, the second sublingual dosage form, labeled as 2, is to be administered under the tongue of the patient and maintained there until fully dissolved; and (c) if the symptoms of anaphylaxis do not improve or terminate within approximately five minutes after administration of the second dose, the third sublingual dosage form, labeled as 3, is to be administered under the tongue of the patient and maintained there until fully dissolved.

The written instructions also provide standard information including the proper storage conditions for the dosage forms, how to properly dispose of the unused dosage forms, contra-indications related to sublingual dosage forms comprising epinephrine, etc.

A carrying case is also included in the kit which provides easy storage for the sublingual dosage forms and also provides additional protection from moisture, light and oxygen.

### Example 5

A Kit Comprising Multiple Sublingual Dosage Forms of Epinephrine for the Treatment of Anaphylaxis

A kit is provided which contains five sublingual dosages forms. The first dose contains 40 mg of epinephrine. The second dose contains 40 mg of epinephrine. The third dose contains about 30 mg of epinephrine. The fourth dose contains 15 mg of epinephrine. The fifth dose contains 7.5 mg of epinephrine. The sublingual dosage forms are packaged in a foil blister pack with numerical markings identifying the order and location of each dose.

Specifically, the foil blister pack containing the five dosage forms is embossed with the numbers 1, 2, 3, 4, and 5, respectively. The numerical markings provide easy identification of each dosage form by the patient. The kit further contains written instructions to aid the patient in administering the dosage forms of epinephrine contained therein in the correct order and at the correct time.

The instructions provide as follows: (a) the first sublingual dosage form, labeled as 1, is to be placed under the tongue of the patient as soon as the patient begins experiencing symptoms of anaphylaxis and maintained there until fully dissolved; (b) if the symptoms of anaphylaxis do not improve or terminate within approximately five minutes, the second sublingual dosage form, labeled as 2, is to be administered under the tongue of the patient and maintained there until fully dissolved; (c) if the symptoms of anaphylaxis do not improve or terminate within approximately five minutes after administration of the second dose, the third sublingual dosage form, labeled as 3, is to be administered under the tongue of the patient and maintained there until fully dissolved; (d) if the symptoms of anaphylaxis do not improve or terminate within approximately five minutes after administration of the third dose, the fourth sublingual dosage form, labeled as 4, is to be administered under the tongue of the patient and maintained there until fully dissolved; and (e) if the symptoms of anaphylaxis do not improve or terminate within approximately five minutes after administration of the fourth dose, the fifth sublingual dosage form, labeled as 5, is to be administered under the tongue of the patient and maintained there until fully dissolved.

The written instructions also provide standard information including the proper storage conditions for the dosage forms, how to properly dispose of the unused dosage forms, contra-indications related to sublingual dosage forms comprising epinephrine, etc.

A carrying case is also included in the kit which provides easy storage for the sublingual dosage forms and also provides additional protection from moisture, light and oxygen.

What is claimed is:

1. A method for treating an allergic emergency in a patient, comprising:
a. administering a first dose of a buccal, lingual or sublingual dosage form comprising epinephrine;
b. administering a second dose of a buccal, lingual or sublingual dosage form comprising epinephrine;
c. optionally administering a third dose of a buccal, lingual or sublingual dosage form comprising epinephrine;
d. optionally administering a fourth dose of a buccal, lingual or sublingual dosage form comprising epinephrine; and
e. optionally administering a fifth dose of a buccal, lingual or sublingual dosage form comprising epinephrine.

2. The method of claim 1, wherein the method comprises administering only said first and second doses.

3. The method of claim 1, wherein the method comprises administering only said first, second and third doses.

4. The method of claim 1, wherein each said buccal, lingual or sublingual dosage form comprises an amount of epinephrine bioequivalent to about 0.01 mg/Kg of epinephrine administered by intramuscular administration.

5. The method of claim 1, wherein each said buccal, lingual or sublingual dosage form comprises an amount of epinephrine bioequivalent to about 0.3 mg of epinephrine administered by intramuscular administration.

6. The method of claim 1, wherein each said buccal, lingual or sublingual dosage form comprises from about 1 mg to about 100 mg of epinephrine.

7. The method of claim 6, wherein each said buccal, lingual or sublingual dosage form comprises from about 15 mg to about 60 mg of epinephrine.

8. The method of claim 1, wherein at least one of said dosage forms is a sublingual tablet.

9. The method of claim 1, wherein each said buccal, lingual or sublingual dosage form further comprises a pharmaceutically acceptable excipient.

10. The method of claim 1, wherein administering said doses is carried out by said patient.

11. The method of claim 1, wherein the time interval between administering each consecutive dose is between about 3 minutes to about 10 minutes.

12. A method for treating an allergic emergency in a patient, comprising
a. administering a first dose of a buccal, lingual or sublingual dosage form comprising epinephrine;
b. administering a second dose of a buccal, lingual or sublingual dosage form comprising epinephrine wherein the amount of epinephrine in said second dose is about 50% to about 100% of the amount of epinephrine in said first dose;
c. optionally administering a third dose of a buccal, lingual or sublingual dosage form comprising epinephrine wherein the amount of epinephrine in said third dose is about 50% to about 100% of the amount of epinephrine in said second dose;
d. optionally administering a fourth dose of a buccal, lingual or sublingual dosage form comprising epinephrine wherein the amount of epinephrine in said fourth dose is about 50% to about 100% of the amount of epinephrine in said third dose; and
e. optionally administering a fifth dose of a buccal, lingual or sublingual dosage form comprising epinephrine wherein the amount of epinephrine in said fifth dose is about 50% to about 100% of the amount of epinephrine in said fourth dose.

13. The method of claim 12, wherein said first dose comprises an amount of epinephrine bioequivalent to about 0.01 mg/Kg of epinephrine administered by intramuscular administration.

14. The method of claim 12, wherein said first dose comprises an amount of epinephrine bioequivalent to about 0.3 mg of epinephrine administered by intramuscular administration.

15. The method of claim 12, wherein said first dose comprises from about 1 mg to about 100 mg of epinephrine.

16. The method of claim 15, wherein said first dose comprises from about 15 mg to about 60 mg of epinephrine.

17. The method of claim 12, wherein at least one of said dosage forms is a sublingual tablet.

18. The method of claim 12, wherein at least one of said buccal, lingual or sublingual dosage forms further comprises a pharmaceutically acceptable excipient.

19. The method of claim 18, wherein the pharmaceutically acceptable excipient comprises an absorption enhancer.

20. The method of claim 19, wherein the absorption enhancer is a transmucosal absorption enhancer.

21. The method of claim 19, wherein the absorption enhancer is present in only said second dose.

22. The method of claim 12, wherein administering said doses is carried out by said patient.

23. The method of claim 12, wherein the time interval between administering each consecutive dose is between about 3 minutes to about 10 minutes.

24. A method for treating an allergic emergency in a patient, comprising
a. administering a first dose of a buccal, lingual or sublingual dosage form comprising epinephrine;
b. administering a second dose of a buccal, lingual or sublingual dosage form comprising epinephrine wherein the amount of epinephrine in said second dose is about 75% to about 125% of the amount of epinephrine in said first dose;
c. optionally administering a third dose of a buccal, lingual or sublingual dosage form comprising epinephrine wherein the amount of epinephrine in said third dose is about 75% to about 125% of the amount of epinephrine in said first dose;
d. optionally administering a fourth dose of a buccal, lingual or sublingual dosage form comprising epinephrine wherein the amount of epinephrine in said fourth dose is about 75% to about 125% of the amount of epinephrine in said first dose; and
e. optionally administering a fifth dose of a buccal, lingual or sublingual dosage form comprising epinephrine wherein the amount of epinephrine in said fifth dose is about 75% to about 125% of the amount of epinephrine in said first dose.

25. The method of claim 24, wherein said first dose comprises an amount of epinephrine bioequivalent to about 0.01 mg/Kg of epinephrine administered by intra-muscular administration.

26. The method of claim 24, wherein said first dose comprises an amount of epinephrine bioequivalent to about 0.3 mg of epinephrine administered by intra-muscular administration.

27. The method of claim 24, wherein said first dose comprises from about 1 mg to about 100 mg of epinephrine.

28. The method of claim 27, wherein said first dose comprises from about 15 mg to about 60 mg of epinephrine.

29. The method of claim 24, wherein at least one of said dosage forms is a sublingual tablet.

30. The method of claim 24, wherein at least one of said buccal, lingual or sublingual dosage forms further comprises a pharmaceutically acceptable excipient.

31. The method of claim 30, wherein the pharmaceutically acceptable excipient comprises an absorption enhancer.

32. The method of claim 31, wherein the absorption enhancer is a transmucosal absorption enhancer.

33. The method of claim 31, wherein the absorption enhancer is present in only said second dose.

34. The method of claim 24, wherein administering said doses is carried out by said patient.

35. The method of claim 24, wherein the time interval between administering each consecutive dose is between about 3 minutes to about 10 minutes.

36. A kit or packaging system for use in the treatment of an allergic emergency, comprising two or more doses of a buccal, lingual or sublingual dosage form comprising epinephrine.

37. The kit or packaging system of claim 36, further comprising written instructions for administering said two or more doses of a buccal, lingual or sublingual dosage form comprising epinephrine.

38. The kit or packaging system of claim 36, wherein said two or more doses of a buccal, lingual or sublingual dosage form comprising epinephrine are contained within a protective liner.

39. The kit or packaging system of claim 38, wherein said protective liner prevents damage due to moisture, light, or oxygen.

40. The kit or packaging system of claim 39, wherein said protective liner is a polymer-lined foil.

41. The kit or packaging system of claim 36, wherein said two or more doses of a buccal, lingual or sublingual dosage form comprising epinephrine are identified by numerical markings or by sequential location within said protective liner to indicate the order of administration of said doses.

42. The kit or packaging system of claim 36, wherein said two or more doses of a buccal, lingual or sublingual dosage form comprising epinephrine are identified in said kit or packaging system by dosage form shape, dosage form size, or dosage form color.

43. A method for treating an allergic emergency in a patient, comprising
a. administering a first dose of a buccal, lingual or sublingual dosage form comprising epinephrine;
b. administering a second dose of a buccal, lingual or sublingual dosage form comprising epinephrine wherein the amount of epinephrine in said second dose is about 50% to about 100% of the amount of epinephrine in said first dose;
c. optionally administering a third dose of a buccal, lingual or sublingual dosage form comprising epinephrine wherein the amount of epinephrine in said third dose is about 50% to about 100% of the amount of epinephrine in said first dose;
d. optionally administering a fourth dose of a buccal, lingual or sublingual dosage form comprising epinephrine wherein the amount of epinephrine in said fourth dose is about 50% to about 100% of the amount of epinephrine in said first dose; and
e. optionally administering a fifth dose of a buccal, lingual or sublingual dosage form comprising epinephrine wherein the amount of epinephrine in said second dose is about 50% to about 100% of the amount of epinephrine in said first dose.

44. A method for treating an allergic emergency in a patient, comprising
a. administering a first dose of a buccal, lingual or sublingual dosage form comprising epinephrine;
b. administering a second dose of a buccal, lingual or sublingual dosage form comprising epinephrine wherein the amount of epinephrine in said second dose is about 75% to about 125% of the amount of epinephrine in said first dose;
c. optionally administering a third dose of a buccal, lingual or sublingual dosage form comprising epinephrine wherein the amount of epinephrine in said third dose is about 75% to about 125% of the amount of epinephrine in said second dose;
d. optionally administering a fourth dose of a buccal, lingual or sublingual dosage form comprising epinephrine wherein the amount of epinephrine in said fourth dose is about 75% to about 125% of the amount of epinephrine in said third dose; and
e. optionally administering a fifth dose of a buccal, lingual or sublingual dosage form comprising epinephrine wherein the amount of epinephrine in said fifth dose is about 75% to about 125% of the amount of epinephrine in said fourth dose.
